SEQUENCE LISTING (4 sequences)

SEQ ID NO: 1 - DNA, 19 bp, Artificial Sequence
  Description of Artificial Sequence Synthetic primer
ggccctaaggaaactgcat

SEQ ID NO: 2 - DNA, 20 bp, Artificial Sequence
  Description of Artificial Sequence Synthetic primer
gggaatgggaatgtaacctg

SEQ ID NO: 3 - protein, 4 aa, Unknown
  Description of Unknown Proline rich domain
PPPY

SEQ ID NO: 4 - protein, 4 aa, Unknown
  Description of Unknown Proline rich domain
PPSY

(19) United States
(12) Patent Application Publication
(10) Pub. No.: US 2022/0088127 A1
(43) Pub. Date: Mar. 24, 2022

(54) TARGETING THE BAG FAMILY FOR THE TREATMENT OF NEURODEGENERATIVE DISEASE

(71) Applicant: Temple University - of the Commonwealth System of Higher Education, Philadelphia, PA (US)

(72) Inventors: Jennifer Gordon, Philadelphia, PA (US); Kamel Khalili, Bala Cynwyd, PA (US); T. Dianne Langford, Bala Cynwyd, PA (US); Taha Mohseni Ahooyi, Philadelphia, PA (US)

(21) Appl. No.: 17/422,628

(22) PCT Filed: Jan. 15, 2020
(86) PCT No.: PCT/US2020/013724

§ 371 Date: Jul. 13, 2021

Related U.S. Application Data
(60) Provisional application No. 62/792,682, filed on Jan. 15, 2019.

(51) Int. Cl. A61K 38/17; A61K 45/06

(52) U.S. Cl. CPC A61K 38/1709; A61K 45/06

(57) ABSTRACT

Compositions in the prevention or treatment of neurodegenerative diseases or disorders associated with human immunodeficiency virus infection include agents which modulate the interactions of HIV Tat and Bcl-2 associated athanogene (BAG) molecules.

### CROSS REFERENCE TO RELATED APPLICATIONS

This application claims the benefit of U.S. Provisional Application 62/792,682 filed on Jan. 15, 2019. The entire contents of this application is incorporated herein by reference in its entirety.

### STATEMENT REGARDING FEDERALLY SPONSORED RESEARCH

This invention was made with government support under Grant Numbers 5P01DA037830, 5P30MH092177 and 5R01HL123093 awarded by the National Institutes of Health. The government has certain rights in the invention.

### TECHNICAL FIELD

Embodiments are directed to compositions in the prevention or treatment of neurodegenerative diseases or disorders associated with human immunodeficiency virus infection. In particular, compositions include agents which modulate the interactions of HIV Tat and Bcl-2 associated athanogene (BAG) molecules. Methods of treatment utilize these compositions.

### BACKGROUND

Accumulation and aggregation of toxic proteins in response to environmental stressors including infection are a critical feature of several neurodegenerative diseases. The heat shock pathway is the first line of defense and is designed for surveillance of proteins post-translationally after cellular insults or stress. Aberrant proteins that cannot be repaired may be targeted to the ubiquitin-proteasome system for degradation. Upon significant accumulation of toxic proteins, the cell undergoes more drastic measures including induction of apoptotic or autophagic processes leading to cell death. Under stress conditions, the inability of the cell to clear abnormal or misfolded proteins resulting in the accumulation of aberrant proteins, such as hyperphosphorylated tau, may lead to disease. The heat shock response works with the ubiquitin proteasome system for the recognition and clearance of aberrant proteins through the binding of a series of chaperone proteins including the heat shock protein 70 (HSP70) for attachment of ubiquitin molecules. Recent studies also confirm important roles for Hsp70 in controlling abnormal tau accumulation by altering function and accumulation of specific tau isoforms. In addition to impacting microtubule formation, dysfunction of the mitochondria is an important mechanism contributing to neuronal loss in AD. Alterations in mitochondria, with an increase in fission and a decrease in fusion, have been reported in neurons of AD brains along with a drop in GTPase activity. Further, interaction between Drp1 and hyperphosphorylated tau has also recently been shown in Alzheimer's Disease brains demonstrating that tau can directly impact mitochondrial function and bioenergetics.

### SUMMARY

The BAG family, including BAG3, through its role in regulating protein quality control, including autophagy and mitophagy, plays a critical role in the regulation of Tau protein, which accumulates in neurological disorders, including neurodegenerative diseases and HIV infection of the brain. Rebalancing of protein quality control through targeting the BAG3 pathway, leads to improvement in clearance of toxic proteins and restoration of normal cellular protein quality control mechanisms and improvement of mitochondrial function in the brain.

Accordingly, in certain embodiments, a composition comprising an agent which inhibits interaction between human immunodeficiency virus (HIV) Tat protein, Bcl-2 associated athanogene (BAG) and/or heat shock protein 70 (HSP70) as measured by a hyperphosphorylated tau accumulation and/or reactive oxygen species in a subject. In certain embodiments, the Bcl-2 associated athanogene (BAG) is Bcl-2 associated athanogene 3 (BAG3). In certain embodiments, the agent comprises small molecules, oligonucleotides, polynucleotides, peptides, polypeptides, proteins, gene-editing agents, siRNA, enzymes, peptide nucleic acids, organic molecules, synthetic molecules or combinations thereof.

In certain embodiments, a method of preventing and/or treating dementia in a subject, comprising: administering to the subject a pharmaceutical composition comprising a therapeutically effective amount of an agent which inhibits interaction between human immunodeficiency virus (HIV) Tat protein, Bcl-2 associated athanogene (BAG) and/or heat shock protein 70 (HSP70), thereby preventing or treating dementia in the subject.

Other aspects are described infra.

### Definitions

The terminology used herein is for the purpose of describing particular embodiments only and is not intended to be limiting of the invention. As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value. Where particular values are described in the application and claims, unless otherwise stated the term “about” meaning within an acceptable error range for the particular value should be assumed.

As used herein, the term “agent” or “candidate therapeutic agent” is meant to encompass any molecule, chemical entity, composition, drug, therapeutic agent, chemotherapeutic agent, or biological agent capable of preventing, ameliorating, or treating a dysfunction or other medical condition. The term includes small molecule compounds, antisense oligonucleotides, siRNA reagents, antibodies, antibody fragments bearing epitope recognition sites, such as Fab, Fab′, F(ab′)₂ fragments, Fv fragments, single chain antibodies, antibody mimetics (such as DARPins, affibody molecules, affilins, affitins, anticalins, avimers, fynomers, Kunitz domain peptides and monobodies), peptoids, aptamers; enzymes, peptides organic or inorganic molecules, natural or synthetic compounds and the like. An agent can be assayed in accordance with the methods of the invention at any stage during clinical trials, during pre-trial testing, or following FDA-approval.

By “antisense oligonucleotides” or “antisense compound” is meant an RNA or DNA molecule that binds to another RNA or DNA (target RNA, DNA). For example, if it is an RNA oligonucleotide it binds to another RNA target by means of RNA-RNA interactions and alters the activity of the target RNA. An antisense oligonucleotide can upregulate or downregulate expression and/or function of a particular polynucleotide. The definition is meant to include any foreign RNA or DNA molecule which is useful from a therapeutic, diagnostic, or other viewpoint. Such molecules include, for example, antisense RNA or DNA molecules, interference RNA (RNAi), micro RNA, decoy RNA molecules, siRNA, enzymatic RNA, short, hairpin RNA (shRNA), therapeutic editing RNA and agonist and antagonist RNA, antisense oligomeric compounds, antisense oligonucleotides, external guide sequence (EGS) oligonucleotides, alternate splicers, primers, probes, and other oligomeric compounds that hybridize to at least a portion of the target nucleic acid. As such, these compounds may be introduced in the form of single-stranded, double-stranded, partially single-stranded, or circular oligomeric compounds.

The term “anti-viral agent” as used herein, refers to any molecule that is used for the treatment of a virus and include agents which alleviate any symptoms associated with the virus, for example, anti-pyretic agents, anti-inflammatory agents, chemotherapeutic agents, and the like. An antiviral agent includes, without limitation: antibodies, aptamers, adjuvants, anti-sense oligonucleotides, chemokines, cytokines, immune stimulating agents, immune modulating agents, B-cell modulators, T-cell modulators, NK cell modulators, antigen presenting cell modulators, enzymes, siRNA's, ribavirin, protease inhibitors, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, or combinations thereof. The term also refers to non-nucleoside reverse transcriptase inhibitors (NNRTIs), nucleoside reverse transcriptase inhibitors (NRTIs), analogs, variants etc.

As used herein, the terms “comprising,” “comprise” or “comprised,” and variations thereof, in reference to defined or described elements of an item, composition, apparatus, method, process, system, etc. are meant to be inclusive or open ended, permitting additional elements, thereby indicating that the defined or described item, composition, apparatus, method, process, system, etc. includes those specified elements—or, as appropriate, equivalents thereof—and that other elements can be included and still fall within the scope/definition of the defined item, composition, apparatus, method, process, system, etc.

As used herein, “Bcl-2 associated athanogene” (BAG) are inclusive of all family members e.g. BAG1, BAG2, BAG3, etc., isoforms, mutants, cDNA sequences, alleles, fragments, species, coding and noncoding sequences, sense and antisense polynucleotide strands, etc.

As used herein “BAG3”, “BAG3 molecules”, “BCL2-associated athanogene 3 (BAG3) genes”, “BCL2-associated athanogene 3 (BAG3) molecules” are inclusive of all family members, isoforms, mutants, cDNA sequences, alleles, fragments, species, coding and noncoding sequences, sense and antisense polynucleotide strands, etc. (HGNC (939) Entrez Gene (9531) Ensembl (ENSG00000151929) OMIM (603883) UniProtKB (095817)). Similarly, “BAG3”, “BAG3 molecules”, “BCL2-associated athanogene 3 (BAG3) molecules” also refer to BAG3 polypeptides or fragment thereof, proteins, variants, derivatives etc. The term “molecule”, thus encompasses both the nucleic acid sequences and amino acid sequences of BAG3.

As used herein, “biological samples” include solid and body fluid samples. The biological samples used in the present invention can include cells, protein or membrane extracts of cells, blood or biological fluids such as ascites fluid or brain fluid (e.g., cerebrospinal fluid). Examples of solid biological samples include, but are not limited to, samples taken from tissues of the central nervous system, bone, breast, kidney, cervix, endometrium, head/neck, gallbladder, parotid gland, prostate, pituitary gland, muscle, esophagus, stomach, small intestine, colon, liver, spleen, pancreas, thyroid, heart, lung, bladder, adipose, lymph node, uterus, ovary, adrenal gland, testes, tonsils, thymus and skin, or samples taken from tumors. Examples of “body fluid samples” include, but are not limited to blood, serum, semen, prostate fluid, seminal fluid, urine, feces, saliva, sputum, mucus, bone marrow, lymph, and tears.

As used herein the phrase “diagnosing” refers to classifying a disease or a symptom, determining a severity of the disease, monitoring disease progression, forecasting an outcome of a disease and/or prospects of recovery. The term “detecting” may also optionally encompass any of the above. Diagnosis of a disease according to the present invention can be effected by determining a level of a polynucleotide or a polypeptide of the present invention in a biological sample obtained from the subject, wherein the level determined can be correlated with predisposition to, or presence or absence of the disease. It should be noted that a “biological sample obtained from the subject” may also optionally comprise a sample that has not been physically removed from the subject.

An “effective amount” as used herein, means an amount which provides a therapeutic or prophylactic benefit.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

By “inhibitory nucleic acid” is meant a double-stranded RNA, siRNA, shRNA, or antisense RNA, or a portion thereof, or a mimetic thereof, that when administered to a mammalian cell results in a decrease (e.g., by 10%, 25%, 50%, 75%, or even 90-100%) in the expression of a target gene. Typically, a nucleic acid inhibitor comprises at least a portion of a target nucleic acid molecule, or an ortholog thereof, or comprises at least a portion of the complementary strand of a target nucleic acid molecule. For example, an inhibitory nucleic acid molecule comprises at least a portion of any or all of the nucleic acids delineated herein.

“Isolated” means altered or removed from the natural state. For example, a nucleic acid or a peptide naturally present in a living animal is not “isolated,” but the same nucleic acid or peptide partially or completely separated from the coexisting materials of its natural state is “isolated.” An isolated nucleic acid or protein can exist in substantially purified form, or can exist in a non-native environment such as, for example, a host cell.

An “isolated nucleic acid” refers to a nucleic acid segment or fragment which has been separated from sequences which flank it in a naturally occurring state, i.e., a DNA fragment which has been removed from the sequences which are normally adjacent to the fragment, i.e., the sequences adjacent to the fragment in a genome in which it naturally occurs. The term also applies to nucleic acids which have been substantially purified from other components which naturally accompany the nucleic acid, i.e., RNA or DNA or proteins, which naturally accompany it in the cell. The term therefore includes, for example, a recombinant DNA which is incorporated into a vector, into an autonomously replicating plasmid or virus, or into the genomic DNA of a prokaryote or eukaryote, or which exists as a separate molecule (i.e., as a cDNA or a genomic or cDNA fragment produced by PCR or restriction enzyme digestion) independent of other sequences. It also includes: a recombinant DNA which is part of a hybrid gene encoding additional polypeptide sequence, complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like.

Unless otherwise specified, a “nucleotide sequence encoding” an amino acid sequence includes all nucleotide sequences that are degenerate versions of each other and that encode the same amino acid sequence. The phrase nucleotide sequence that encodes a protein or an RNA may also include introns to the extent that the nucleotide sequence encoding the protein may in some version contain an intron(s).

“Parenteral” administration of an immunogenic composition includes, e.g., subcutaneous (s.c.), intravenous (i.v.), intramuscular (i.m.), or intrasternal injection, or infusion techniques.

The terms “patient” or “individual” or “subject” are used interchangeably herein, and refers to a mammalian subject to be treated, with human patients being preferred. In some cases, the methods of the invention find use in experimental animals, in veterinary application, and in the development of animal models for disease, including, but not limited to, rodents including mice, rats, and hamsters, and primates.

The terms “pharmaceutically acceptable” (or “pharmacologically acceptable”) refer to molecular entities and compositions that do not produce an adverse, allergic or other untoward reaction when administered to an animal or a human, as appropriate. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance.

The term “polynucleotide” is a chain of nucleotides, also known as a “nucleic acid” or “nucleic acid sequence” and include, but are not limited to, all nucleic acid sequences which are obtained by any means available in the art, both naturally occurring and synthetic nucleic acids, complementary DNA (cDNA), linear or circular oligomers or polymers of natural and/or modified monomers or linkages, including deoxyribonucleosides, ribonucleosides, substituted and alpha-anomeric forms thereof, peptide nucleic acids (PNA), locked nucleic acids (LNA), phosphorothioate, methylphosphonate, and the like. The nucleic acid sequences may be “chimeric,” that is, composed of different regions. In the context of this invention “chimeric” compounds are oligonucleotides, which contain two or more chemical regions, for example, DNA region(s), RNA region(s), PNA region(s) etc. Each chemical region is made up of at least one monomer unit, i.e., a nucleotide. These sequences typically comprise at least one region wherein the sequence is modified in order to exhibit one or more desired properties.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “target nucleic acid” sequence refers to a nucleic acid (often derived from a biological sample), to which the oligonucleotide is designed to specifically hybridize. The target nucleic acid has a sequence that is complementary to the nucleic acid sequence of the corresponding oligonucleotide directed to the target. The term target nucleic acid may refer to the specific subsequence of a larger nucleic acid to which the oligonucleotide is directed or to the overall sequence (e.g., gene or mRNA). The difference in usage will be apparent from context.

The term “transfected” or “transformed” or “transduced” means to a process by which exogenous nucleic acid is transferred or introduced into the host cell. A “transfected” or “transformed” or “transduced” cell is one which has been transfected, transformed or transduced with exogenous nucleic acid. The transfected/transformed/transduced cell includes the primary subject cell and its progeny.

To “treat” a disease as the term is used herein, means to reduce the frequency or severity of at least one sign or symptom of a disease or disorder experienced by a subject. Treatment of a disease or disorders includes the eradication of a virus.

“Treatment” is an intervention performed with the intention of preventing the development or altering the pathology or symptoms of a disorder. Accordingly, “treatment” refers to both therapeutic treatment and prophylactic or preventative measures. “Treatment” may also be specified as palliative care. Those in need of treatment include those already with the disorder as well as those in which the disorder is to be prevented. Accordingly, “treating” or “treatment” of a state, disorder or condition includes: (I) eradicating the virus; (2) preventing or delaying the appearance of clinical symptoms of the state, disorder or condition developing in a human or other mammal that may be afflicted with or predisposed to the state, disorder or condition but does not yet experience or display clinical or subclinical symptoms of the state, disorder or condition; (3) inhibiting the state, disorder or condition, i.e., arresting, reducing or delaying the development of the disease or a relapse thereof (in case of maintenance treatment) or at least one clinical or subclinical symptom thereof; or (4) relieving the disease, i.e., causing regression of the state, disorder or condition or at least one of its clinical or subclinical symptoms. The benefit to an individual to be treated is either statistically significant or at least perceptible to the patient or to the physician.

The term “variant,” when used in the context of a polynucleotide sequence, may encompass a polynucleotide sequence related to a wild type gene. This definition may also include, for example, “allelic,” “splice,” “species,” or “polymorphic” variants. A splice variant may have significant identity to a reference molecule, but will generally have a greater or lesser number of polynucleotides due to alternate splicing of exons during mRNA processing. The corresponding polypeptide may possess additional functional domains or an absence of domains. Species variants are polynucleotide sequences that vary from one species to another. Of particular utility in the invention are variants of wild type gene products. Variants may result from at least one mutation in the nucleic acid sequence and may result in altered mRNAs or in polypeptides whose structure or function may or may not be altered. Any given natural or recombinant gene may have none, one, or many allelic forms. Common mutational changes that give rise to variants are generally ascribed to natural deletions, additions, or substitutions of nucleotides. Each of these types of changes may occur alone, or in combination with the others, one or more times in a given sequence.

A “vector” is a composition of matter which comprises an isolated nucleic acid and which can be used to deliver the isolated nucleic acid to the interior of a cell. Examples of vectors include but are not limited to, linear polynucleotides, polynucleotides associated with ionic or amphiphilic compounds, plasmids, and viruses. Thus, the term “vector” includes an autonomously replicating plasmid or a virus. The term is also construed to include non-plasmid and non-viral compounds which facilitate transfer of nucleic acid into cells, such as, for example, polylysine compounds, liposomes, and the like. Examples of viral vectors include, but are not limited to, adenoviral vectors, adeno-associated virus vectors, retroviral vectors, and the like.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

Where any a nucleic acid sequence or an amino acid sequence is specifically referred to by a Swiss Prot. or GENBANK Accession number, the sequence is incorporated herein by reference. Information associated with the accession number, such as identification of signal peptide, extracellular domain, transmembrane domain, promoter sequence and translation start, is also incorporated herein in its entirety by reference.

All genes, gene names, and gene products disclosed herein are intended to correspond to homologs from any species for which the compositions and methods disclosed herein are applicable. Thus, the terms include, but are not limited to genes and gene products from humans and mice. It is understood that when a gene or gene product from a particular species is disclosed, this disclosure is intended to be exemplary only, and is not to be interpreted as a limitation unless the context in which it appears clearly indicates. Thus, for example, for the genes disclosed herein, which in some embodiments relate to mammalian nucleic acid and amino acid sequences are intended to encompass homologous and/or orthologous genes and gene products from other animals including, but not limited to other mammals, fish, amphibians, reptiles, and birds. In preferred embodiments, the genes or nucleic acid sequences are human.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIGS. 1A and 1B is a Western blot (FIG. 1A) and a graph (FIG. 1B) demonstrating that hp-tau is increased in HIVE and AD brain tissue. FIG. 1A: Is a representative Western blot showing soluble hp-tau (s262) and total tau (HT7) in age-matched controls (normal), AD Braak stages 1, 3, 5 (Br1, 3, 5), and HIV encephalitis (HIVE) patients. FIG. 1B: Is a graphic representation of percent total tau that is hyperphosphorylated (hp-tau/total tau) in each case compared to control.

FIGS. 2A-2C is a Western blot (FIG. 2A) and a series of graphs (FIG. 2B, FIG. 2C) demonstrating that induction of Tat in the Tat transgenic mouse model induces increased levels of hp-tau. FIG. 2A: Western blot of frontal cortex from Tat-Tg mice +Dox (n=4) and Tat-tg −Dox (n=3). FIG. 2B: hp-tau s396 levels/total tau (HT7) relative to loading control (*p=0.0348). FIG. 2C: total tau (HT7) relative to loading control (p=0.1585). NS, not significant.

FIGS. 3A, 3B are blots demonstrating suppression of BAG3 by Tat in neuronal cells. To characterize potential effects of Tat on protein quality control (PQC), neuronal cells were transduced with adenovirus encoding Tat or control (Ad-Tat vs. Ad-Null). Primary hippocampal (FIG. 3A) or cortical (FIG. 3B) neuronal cells transduced with Ad-Tat showed a substantial decrease in levels of BAG3 which was sustained over 5 days in culture. These results link Tat to regulation of protein quality control and autophage mediated by BAG3 in neuronal cells.

FIG. 4 is a blot demonstrating the suppression of BAG3 in transgenic animals expressing Tat.

FIGS. 5A, 5B are photographs of immunocytostains showing that Tat disrupts co-localization of BAG3 and HSP70 in neuronal cells.

FIGS. 6A, 6B are Western blots demonstrating the expression of ubiquitinated proteins in soluble and insoluble fractions of cells expressing BAG3, siBAG3, or control GFP proteins.

FIGS. 7A and 7B are immunocytostains demonstrating the co-localization of BAG3 and Tom20 in cells expressing Tat. Immunocytochemistry shows in the absence of Tat, BAG3 and Tom20 are found in the cytoplasm and co-localize in the perinuclear region. In the presence of Tat, large numbers of puncta containing both BAG3 and Tom20 are seen throughout the cytoplasm, showing changes in localization of BAG3 and Tom20 in the cells under stress induced by the presence of Tat.

FIGS. 8A and 8B are immunocytostains demonstrating that Tat effects subcellular co-location of BAG3 with Parkin in hippocampal neurons. Immunocytochemistry shows BAG3 localization to both the cytoplasmic and nuclear compartments. Co-localization of Parkin and BAG3 in the cytoplasm of neuronal cells can be seen, more specifically localizing to puncta in the perinuclear region (FIG. 8A). This colocalization is disrupted upon treatment with 50 nM of recombinant Tat (rTat).

FIG. 9 is a blot showing the effect of BAG3 on mitophagy by regulating PINK1 and Parkin levels. Adenovirus was used to deliver siRNA targeting BAG3 (AdsiBAG3) in neuronal cells in order to knock down BAG3 levels at increasing multiplicity of infection (MOI). Silencing of BAG3 profoundly decreased the levels of Pink1 and Parkin, which protect cells from stress-induced mitochondrial dysfunction and mitochondrial autophagy (mitophagy). Tom20 levels remained unchanged. This data demonstrates the ability of BAG3 to control PQC and mitophagy by modulating levels by acting through PINK1 and Parkin.

FIG. 10 is an analysis of the mitochondrial network in neurons undergoing rTat treatment: Top: immunofluorescent images of hippocampal neurons probed with Tom20 to study the mitochondrial network in control (left) and neurons treated with 50 nM rTat protein (right). Middle: image processing of the micrographs in the top panel reveals that rTat disturbs the normal concentration of mitochondria at the axon hillock (left) to a rather uniformly distributed form (right). MATLAB® image processing was utilized to estimate the point-wise density of the red channel. Bottom: contour plots of the red channel density quantitatively confirms the alterations in the mitochondrial network morphology induced by rTat.

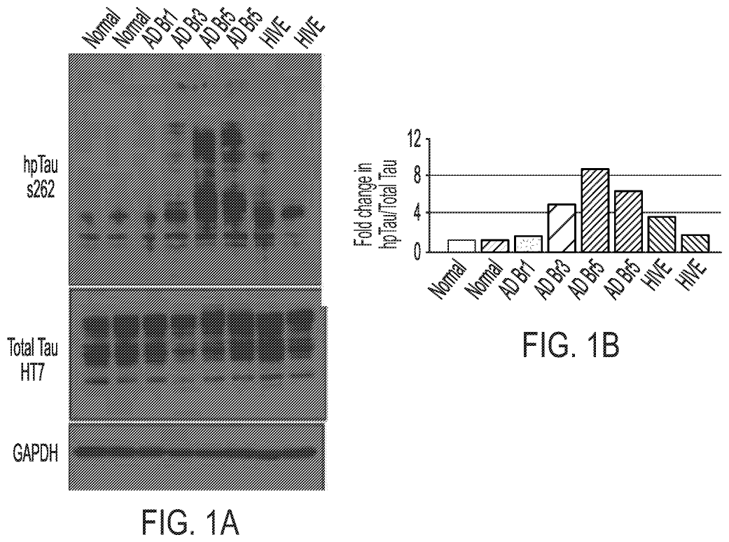

FIG. 1I demonstrates that HIV-1 Tat promotes the initiation of mitophagy signaling by increasing levels of mitophagy-associated proteins. Primary neurons were treated with Tat (50 nM) and subfractioned by differential centrifugation to assess sub-cellular localization. As seen, in the presence of Tat, Parkin (PARK2) immunoreactivity was increased in the mitochondrial fraction. Examination of PINK1 levels by Western blot showed a modest increase in the level of proteins corresponding to two isoforms of PINK1 CoxIV was used as a mitochondrial loading control and tubulin was used as a cytosolic loading).

FIG. 12 is a graph demonstrating that HIV-Tat reduces ATP generation.

FIG. 13 is a graph demonstrating that HIV-Tat increases mtDNA lesions and inhibits SOD activity. Treatment of primary neuronal cells with HIV-1 Tat induced mitochondrial DNA (mtDNA) damage as detected by a decrease in the intensity of the amplified 10 k DNA fragment from mtDNA, which correlates with a decrease in the activity of superoxide dismutase (SOD), indicative of reduced antioxidant capacity and increased susceptibility to superoxide-mediated stress.

FIGS. 14A-14F demonstrate the effect of Tat on hippocampal neurons in microelectrode array (MEA). FIG. 14: MEA neuronal cells cultured 2 days in vitro (top) and 16 days in vitro (bottom). FIG. 14B: Extracellular recordings of neuronal cultures Ad-Null (control) and Ad-Tat (Tat). FIG. 14C: Raster plots of the detected spikes of neuronal cultures treated with Ad-Null or Ad-Tat. FIG. 14D: Normalized mean peak WPR. FIG. 14E: Normalized mean peak absolute amplitudes. FIG. 14F: Normalized firing frequencies. The quantified MEA data show the detrimental effects of Tat expression on the neuronal firing activity in terms of the frequency amplitude and wave propagation rates. Quantified data show mean±StDev. For FIGS. 14B through 14F, all control neuronal cultures (Ad-Null) are shown in blue graphs, and Tat-treated neuronal cultures (Ad-Tat) are shown in red graphs.

FIG. 15 shows the effect of BAG3 overexpression and knock-down on neuronal activity as recorded by microelectrode array (MEA). Top: BAG3 overexpression induces irregularities in neuronal spiking including increased firing frequency and amplitude in the course of 4 days of transduction. Bottom. siRNA-mediated BAG3 knock-down significantly attenuates the neuronal electro-physiological signaling within 2 days. This effect emerges in both the firing frequency and firing amplitude and worsens within the course of 4 days post-transduction.

FIG. 16 shows the effect of Tat expression and BAG3 overexpression on neuronal activity by microelectrode array (MEA). Tat expression (bottom panels) completely silences neuronal activity as compared to control (top panels) within the course of 4 days post-transduction. BAG3 overexpression (Ad-BAG3) partially restores the neuronal firing within two days of transduction in Tat expressing cells (day 8, bottom panel).

FIG. 17 shows the effect of Tat in hippocampal slices from Tat transgenic mice as recorded by MEA. MEA electrophysiological recordings in the absence of Tat (−Dox) and in presence of Tat (+Dox). As indicated by the recordings, 72 hours of Dox-mediated Tat expression has detrimental effects on hippocampal signal transmission in terms of firing frequency and amplitude. Further network analysis reveals Tat's adverse effects on communication between different hippocampal regions.

FIGS. 18A-18F are a series images from immune stains and blots demonstrating that HIV-1 Tat promotes alterations in synaptic vesicle proteins distribution and homeostasis. FIG. 18A: Single neuron images showing that Tat overexpression affects neuronal processes and synaptic vesicle distribution as stained with Tubb3 and synapsins (Syn), respectively. Synapsins accumulate close to soma upon Tat expression, compared to the control where synapsins are primarily observed in neuronal processes. FIG. 18B: Synapsin distribution in neurons in a population wide image shows the disruption of synaptic vesicle network during Tat expression. FIG. 18C: A second synaptic vesicle marker, synaptotagmin 1 (Syt1), confirms the aggregation of synaptic vesicle proteins in neurons upon Tat expression as compared to the Ad-Null transduction. FIG. 18D: In addition to distribution, total synapsin proteins levels are altered in the soluble and insoluble fractions of neuronal lysate upon Tat expression. FIG. 18E: Synaptotagmin 1 exhibits more accumulation in the insoluble fraction compared to soluble fraction (˜70%) in neurons where Tat is expressed. FIG. 18F: Dose response of soluble recombinant Tat (rTat) protein on the distribution of synapsins indicates the increased impairment at higher doses of Tat. Syt1, Syn, and Tubb3 stand for synaptotagmin 1, synapsins, and β3-tubulin, respectively.

FIGS. 19A-19J are a series of graphs and blots demonstrating thatHIV-1 Tat decreases BAG3 protein and mRNA. FIG. 19A: Tat expression reduced BAG3 protein in rat primary hippocampal and cortical neurons, R1 and R2 indicate two replicates shown on this gel. FIGS. 19B, 19C: Hippocampal and cortical neurons expressing Tat show time-dependent reduction in BAG3 (VCP was probed as a control). FIG. 19D: BAG3 level drops in the brains of Tat-transgenic mice under Dox-induced Tat expression. FIG. 19E: Tat expression results in a decreased level of BAG3 protein even during adenoviral overexpression of BAG3, indicating a post-transcriptional reduction. FIG. 19F: Inhibition of the proteasome using MG132 does not restore Tat-induced BAG3 reduction. FIG. 19G: Inhibition of the lysosome using Baf A1 does not block Tat-induced BAG3 decrease. FIG. 19H: Neuronal BAG3 mRNA is decreased by Tat expression. FIG. 19I: qRT-PCR measurement of BAG3 mRNA levels under control and Tat expression when transcription is inhibited by Actinomycin D. FIG. 19J: Recombinant Tat decreases BAG3 mRNA levels. Quantifications show mean±SD with n≥3. P-values calculated using t-test where *P≤0.01, **P≤0.005, ***P≤0.001.

FIGS. 20A-20F are a series of immunostains and blots demonstrating that BAG3 KD leads to the accumulation and aggregation of synaptic vesicle proteins. FIG. 20A: BAG3 co-localizes with Syt1 in the control and oxidative-stress induced neurons. FIG. 20B: SVs probed with Syn and Syt1 indicate the accumulation of synaptic vesicles along the major neuronal processes and soma under BAG3 KD. FIG. 20C: BAG3 KD under normal and H₂O₂-induced oxidative stress conditions results in accumulation of Syn in the insoluble fraction of neuronal protein lysate. FIG. 20D: BAG3 KD under normal and stress conditions results in accumulation of Syt1 in the insoluble fraction of neuronal protein lysate. FIG. 20E: Neuronal culture in a microfluidic device allows the assessment of neuronal processes separate from the soma. BAG3 KD using Ad-siBAG3 transduction leads to the increased formation and accumulation of syn-positive aggregates along processes. FIG. 20F: BAG3 KD impairs the distribution of synapsins in the neurons as compared to the control.

FIGS. 21A-21I are a series of blots, immunostains and a graph demonstrating that BAG3 KD impairs lysosomal autophagy and client protein ubiquitination under oxidative stress. FIG. 21A: Under control and oxidative stress conditions, BAG3 KD leads to decreased level of LC3-II. FIG. 21B: Co-immunoprecipitation with BAG3 and LC3 confirms the interaction of BAG3 and LC3. FIG. 21C: Under control and oxidative stress conditions, BAG3 KD leads to reduced levels of ATG5, a protein essential for LC3-4 to LC3-II conversion. FIG. 21D: Co-immunoprecipitation with ATG5 antibody and probing for BAG3 confirms the interaction of BAG3 and ATG5. FIG. 21E: BAG3 KD in neurons leads to inhibition of ubiquitination under oxidative stress and inhibition of proteasome or lysosome. The bracket shows the higher molecular weight region at which ubiquitination is impaired under BAG3 KD. FIG. 21F: ATG5 co-localizes with Hsc70 in neurons. FIG. 21G: BAG3 co-localizes with UB in neurons. FIG. 21H: ATG5 and BAG3 are suppressed under Tat expression over a range of 2-4 days post-transduction. FIG. 21I: ATG5 mRNA level is not decreased by Tat expression compared to control.

FIGS. 22A-22E are a series of blots and images of stains demonstrating that BAG3 interacts with synaptic vesicle proteins and synapsins likely via the WW domain. FIG. 22A: Co-immunoprecipitation with Syn antibody and probing for BAG3 confirms the interaction of BAG3 with synapsins in rat primary cortical and hippocampal neurons under normal and oxidative stress conditions. FIG. 22B: Co-immunoprecipitation with BAG3 antibody and probing for synaptotagmin 1 confirms the interaction of BAG3 and Syt1. FIG. 22C: BAG3 co localizes with Syt1 in neurons. FIGS. 22D, 22E: Expression of Flag-BAG3 (1-300) in neurons results in the disruption of neuronal synapsins distribution similar to BAG3 KD and Tat expression.

FIG. 23 is a schematic representation depicting BAG3-mediated homeostasis of synaptic vesicle proteins turnover disrupted by HIV-1 Tat. BAG3 is essential for the formation of LC3-II via regulating ATG5 protein. BAG3 is involved in the homeostasis and turnover of synapsins through lysosmal autophagy and vesicle axonal transport.

FIG. 24A is a series of immunohistochemistry images showing BAG3 downregulation in the brain slices from Tg26 mice. FIG. 24B is a blot showing the Ad-Tat transduction of primary neurons causes accumulation of Syt1 in insoluble neuronal proteins fraction.

FIGS. 25A-25D are a series of blots, images of stains and plots demonstrating that BAG3 overexpression partially restores synaptic vesicles distribution and neuronal activity. FIG. 25A: BAG3 overexpression restores the impairing effect of HIV-1 Tat on SVs distribution in immunocytochemical images as probed with synapsin antibody. FIG. 25B. BAG3 overexpression lowers Tat expression in neurons. FIGS. 25C and 25D: BAG3 overexpression restores the neuronal activity as measured by microelectrode arrays.

FIG. 26A is a graph demonstrating the Syt1 protein levels in the insoluble fraction increased as a result of Tat expression in primary rat neurons. FIG. 26B is a graph showing the ATG5 protein levels in primary rat decreased under BAG3 KD in the presence and absence of protein degradation inhibitors and oxidative stress. FIG. 26C is a series of images of stains demonstrating that BAG3 KD leads to the accumulation of synapsin along axon grown in microfluidic channels.

### DETAILED DESCRIPTION

The following description of the various embodiments is merely exemplary in nature and is in no way intended to limit the invention, its application or uses. Embodiments of the invention may be practiced without the theoretical aspects presented. Moreover, the theoretical aspects are presented with the understanding that Applicants do not seek to be bound by the theory presented.

Human Immunodeficiency Virus (HIV)

Viral infections, such as human immunodeficiency virus (HIV), can be a significant source of cell stress on neuronal cells. A large number of HIV-1 positive individuals exhibit progressive neurological disorders even under conditions when the virus is well controlled with antiretroviral therapy (ART). Results from several laboratories established the ability of the HIV regulatory protein, Tat, to trigger pathways that lead to neuronal dysfunction and injury including energy metabolism. Tat possesses neurotoxic activity, one mechanism by which may be inflicting stress and increasing the levels of cytosolic and/or mitochondrial reactive oxygen species (ROS) leading to cellular and mitochondrial damage, transcriptional and post-transcriptional dysregulation including premature degradation and misfolding of proteins, cytoplasmic and nuclear autophagy, as well as mitochondrial dysfunction and decreases in oxidative phosphorylation (OxPhos) efficiency.

The genetic variability of HIV is reflected in the multiple groups and subtypes that have been described. A collection of HIV sequences is compiled in the Los Alamos HIV databases and compendiums (hiv.lanl.gov). The methods and compositions of the invention can be applied to HIV from any of those various groups, subtypes, and circulating recombinant forms. These include for example, the HIV-1 major group (often referred to as Group M) and the minor groups, Groups N, O, and P, as well as but not limited to, any of the following subtypes, A, B, C, D, F, G, H, J and K. or group (for example, but not limited to any of the following Groups, N, O and P) of HIV.

The HIV genome consists of two identical single-stranded RNA molecules that are enclosed within the core of the virus particle. The genome of the HIV provirus also known as proviral DNA, is generated by the reverse transcription of the viral RNA genome into DNA, degradation of the RNA and integration of the double-stranded HIV DNA into the human genome. The DNA genome is flanked at both ends by LTR (long terminal repeat) sequences. The 5′ LTR region codes for the promotor for transcription of the viral genes. In the direction 5′ to 3′ the reading frame of the gag gene follows, encoding the proteins of the outer core membrane (MA, p17), the capsid protein (CA, p24), the nucleocapsid (NC, p7) and a smaller, nucleic acid-stabilizing protein. The gag reading frame is followed by the pol reading frame coding for the enzymes protease (PR, p12), reverse transcriptase (RT, p51) and RNase H (p15) or RT plus RNase H (together p66) and integrase (IN, p32). Adjacent to the pol gene, the env reading frame follows from which the two envelope glycoproteins gp120 (surface protein, SU) and gp41 (transmembrane protein, TM) are derived. In addition to the structural proteins, the HIV genome codes for several regulatory proteins: Tat (transactivator protein) and Rev (RNA splicing-regulator) are necessary for the initiation of HIV replication, while the other regulatory proteins Nef (negative regulating factor), Vif (viral infectivity factor), Vpr (virus protein r) and Vpu (virus protein unique) have an impact on viral replication, virus budding and pathogenesis. HIV-2 codes for Vpx (virus protein x) instead of Vpu, which is partially responsible for the reduced pathogenicity of HIV-2.

Tat accelerates the availability of viral RNA for virus production approximately 100-fold. Tat binds to the TAR sequence of viral RNA, but not to cellular RNA. Furthermore, Tat is able to transactivate additional HIV genomes present in the cell. Tat expression is induced by Tat but also by cytokines such as p65 and NFκB.

Bcl-2 Associated Athanogene (BAG) Family

The BAG (Bcl-2 associated athanogene) family is a multifunctional group of proteins that perform diverse functions ranging from apoptosis to tumorigenesis. An evolutionarily conserved group, these proteins are distinguished by a common conserved region known as the BAG domain. BAG genes have been found in yeasts, plants, and animals, and are believed to function as adapter proteins forming complexes with signaling molecules and molecular chaperones.

BAG proteins are characterized by a common conserved region located near the C terminus, termed the BAG domain (BD) that mediates direct interaction with the ATPase domain of Hsp70/Hsc70 molecular chaperones. Six BAG family members have been identified in humans and shown to regulate, both positively and negatively, the function of Hsp70/Hsc70, and to form complexes with a range of transcription factors modulating various physiological processes including apoptosis, tumorigenesis, neuronal differentiation, stress responses, and the cell cycle. In addition to the conserved BD, several other domains within the BAG proteins have been identified and are likely to modulate both target specificity and BAG protein localization within cells. The BAG proteins generally differ in the N-terminal region, which imparts specificity to particular proteins and pathways. Ubiquitin-like domain at the N terminus of human BAG proteins (BAG1 and BAG6) is probably functionally relevant and conserved in yeast, plants, and worms. BAG proteins regulate diverse physiological processes in animals, including apoptosis, tumorigenesis, neuronal differentiation, stress responses, and the cell cycle

The six human BAG proteins identified are BAG-1 (RAP46/HAP46), BAG-2, BAG-3 (CAIR-1/Bis), BAG-4 (SODD), BAG-5, and BAG-6 (BAT3/Scythe), and all share the signature BD near the C-terminal end, with the exception of BAG 5, which contains four of such domains.

Compositions

It was demonstrated herein, that in the presence of Tat, several factors associated with pro-survival and pro-apoptosis are affected. Among them, BAG3 protein which is a co-chaperone/partner of HSP70 and is involved in the removal of dysfunctional and obsolete organelles including mitochondria through a process called mitophagy, and participates in autophagy and clearance of damaged and misfolded proteins by the protein quality control (PQC) pathway. Thus, Tat may inflict damage to proteins and organelles by inducing ROS production, which prevents the repair and/or removal of inefficient and toxic proteins from the cell environment by interfering with BAG3 function, all of which results in neuronal cell injury and damage.

Taken together, the HIV Tat protein and BAG-3 can be linked to the accumulation of hyperphosphorylated tau and, ultimately, synergistic effects in their impact on neuronal bioenergetics and survival. For example, the HIV protein Tat has been shown to induce hp-tau in human primary neurons in vitro and in the brains of Tat transgenic mice in vivo. In addition, studies show that in the frontal cortex of HIVE patients, hyperphosphorylated tau levels are also increased. The strong association between Tat, BAG3 and HSP70, as well as between HSP70 and tau, provides evidence for a molecular cross-talk between these two pathways which ultimately controls tau accumulation and clearance, as well as mitochondrial bioenergetic function in neurons.

Bcl-2 associated anthanogene-3 (BAG3), also known as BCL2-Associated Athanogene 3; MFM6; Bcl-2-Binding Protein Bis; CAIR-1; Docking Protein CAIR-1; BAG Family Molecular Chaperone Regulator 3; BAG-3; BCL2-Binding Athanogene 3; or BIS, is a cytoprotective polypeptide that competes with Hip-1 for binding to HSP 70. The NCBI reference amino acid sequence for BAG3 can be found at Genbank under accession number NP_004272.2; Public GI:14043024. The NCBI reference nucleic acid sequence for BAG3 can be found at Genbank under accession number NM_004281.3 GI:62530382. Other BAG3 amino acid sequences include, for example, without limitation, 095817.3 GI:12643665; EAW49383.1 GI:119569768; EAW49382.1 GI:119569767; and CAE55998.1 GI:38502170. The BAG3 polypeptide of the invention can be a variant of a polypeptide described herein, provided it retains functionality.

In certain embodiments, a composition comprises an agent which inhibits interaction between human immunodeficiency virus (HIV) Tat protein, BAG and/or heat shock protein 70 (HSP70) as measured by a hyperphosphorylated tau accumulation and/or reactive oxygen species in a subject. In certain embodiments, the agent comprises small molecules, oligonucleotides, polynucleotides, peptides, polypeptides, proteins, gene-editing agents, siRNA, enzymes, peptide nucleic acids, organic molecules, synthetic molecules or combinations thereof. In certain embodiments, the BAG molecule is BAG3.

Although the invention includes all BAG molecules, BAG3 will be used to illustrate the invention and usage of the term “BAG3” is not intended to limit the invention in any way.

Antisense BAG or Tat Oligonucleotides: In certain embodiments, a therapeutic agent for treatment of subjects increases the expression or amounts of BAG, e.g. BAG3 in a cell. In some embodiments, compositions comprise nucleic acid sequences complementary to BCL2-associated athanogene 3 (BAG3), including without limitation, cDNA, sense and/or antisense sequences of BAG3.

In another embodiment, it may be necessary to increase the expression of BAG3 comprising one or more variants in a cell or patient by oligonucleotides that modulate the expression of BAG3, for example, transcriptional regulator elements. In a preferred embodiment, an oligonucleotide comprises at least five consecutive bases complementary to a nucleic acid sequence, wherein the oligonucleotide specifically hybridizes to a target sequence and modulates expression of BAG3 in vivo or in vitro. In another preferred embodiment, the oligonucleotides include variants in which a different base is present at one or more of the nucleotide positions in the compound. For example, if the first nucleotide is an adenosine, variants may be produced which contain thymidine, guanosine or cytidine at this position. This may be done at any of the positions of the oligonucleotide. These compounds are then tested using the methods described herein to determine their ability to inhibit expression of a target nucleic acid.

In certain embodiments, a therapeutic agent for treatment of subjects decreases the expression or amounts of Tat in a cell. In some embodiments, compositions comprise inhibitory nucleic acids complementary to Tat and/or antisense oligonucleotides which hybridize to BCL2-associated athanogene (BAG) nucleic acid sequences which increase the expression of BAG, e.g. BAG3.

In some embodiments, homology, sequence identity or complementarity, between the oligonucleotide and target is from about 50% to about 60%. In some embodiments, homology, sequence identity or complementarity, is from about 60% to about 70%. In some embodiments, homology, sequence identity or complementarity, is from about 70% to about 80%. In some embodiments, homology, sequence identity or complementarity, is from about 80% to about 90%. In some embodiments, homology, sequence identity or complementarity, is about 90%, about 92%, about 94%, about 95%, about 96%, about 97%, about 98%, about 99% or about 100%.

In another preferred embodiment, an oligonucleotide comprises combinations of phosphorothioate internucleotide linkages and at least one internucleotide linkage selected from the group consisting of: alkylphosphonate, phosphorodithioate, alkylphosphonothioate, phosphoramidate, carbamate, carbonate, phosphate triester, acetamidate, carboxymethyl ester, and/or combinations thereof.

In certain embodiments of the present invention oligomeric oligonucleotides, particularly oligonucleotides, bind to target nucleic acid molecules and modulate the expression of molecules encoded by a BAG3 gene comprising one or more variants that would either benefit from an increase in BAG3 or whether deletion, substitution or some other mechanism wherein the agent used would be therapeutically beneficial to that particular subject.

In embodiments of the present invention oligomeric oligonucleotides, particularly oligonucleotides, bind to target nucleic acid molecules and modulate the expression of molecules encoded by a tat gene comprising one or more variants that decrease expression of Tat or whether deletion, substitution or some other mechanism wherein the agent used would be therapeutically beneficial to that particular subject.

The oligonucleotides, include, antisense oligomeric compounds, antisense oligonucleotides, external guide sequence (EGS) oligonucleotides, alternate splicers, primers, probes, and other oligomeric compounds that hybridize to at least a portion of the target nucleic acid. As such, these compounds may be introduced in the form of single-stranded, double-stranded, partially single-stranded, or circular oligomeric compounds.

Targeting an oligonucleotide to a particular nucleic acid molecule, in the context of this invention, can be a multistep process. The process usually begins with the identification of a target nucleic acid whose function is to be modulated. This target nucleic acid may be, for example, a cellular gene (or mRNA transcribed from the gene) whose expression is associated with a particular disorder or disease state.

The targeting process usually also includes determination of at least one target region, segment, or site within the target nucleic acid for the antisense interaction to occur such that the desired effect, e.g., modulation of expression, will result. Within the context of the present invention, the term “region” is defined as a portion of the target nucleic acid having at least one identifiable structure, function, or characteristic. Within regions of target nucleic acids are segments. “Segments” are defined as smaller or sub-portions of regions within a target nucleic acid. “Sites,” as used in the present invention, are defined as positions within a target nucleic acid.

In another preferred embodiment, the antisense oligonucleotides bind to coding and/or non-coding regions of a target polynucleotide e.g. BAG3, Tat and modulate the expression and/or function of the target molecule.

In another preferred embodiment, the antisense oligonucleotides bind to natural antisense polynucleotides and modulate the expression and/or function of the target molecule. An example of a “function” can be one which inhibits a negative regulator of transcription, thus allowing for an increased expression of a desired molecule, such as, for example, BAG3. In certain embodiments, the antisense oligonucleotides inhibit the expression or activity of Tat.

In another preferred embodiment, the antisense oligonucleotides bind to sense polynucleotides and modulate the expression and/or function of the target molecule e.g. increase BAG3 or decrease Tat.

In embodiments of the invention the oligonucleotides bind to an antisense strand of a particular target. The oligonucleotides are at least 5 nucleotides in length and can be synthesized so each oligonucleotide targets overlapping sequences such that oligonucleotides are synthesized to cover the entire length of the target polynucleotide. The targets also include coding as well as non-coding regions.

Antisense compounds include antisense oligonucleotides, ribozymes, external guide sequence (EGS) oligonucleotides, siRNA compounds, single- or double-stranded RNA interference (RNAi) compounds such as siRNA compounds, and other oligomeric compounds which hybridize to at least a portion of the target nucleic acid and modulate its function. As such, they may be DNA, RNA, DNA-like, RNA-like, or mixtures thereof, or may be mimetics of one or more of these. These compounds may be single-stranded, double-stranded, circular or hairpin oligomeric compounds and may contain structural elements such as internal or terminal bulges, mismatches or loops. Antisense compounds are routinely prepared linearly but can be joined or otherwise prepared to be circular and/or branched. Antisense compounds can include constructs such as, for example, two strands hybridized to form a wholly or partially double-stranded compound or a single strand with sufficient self-complementarity to allow for hybridization and formation of a fully or partially double-stranded compound. The two strands can be linked internally leaving free 3′ or 5′ termini or can be linked to form a continuous hairpin structure or loop. The hairpin structure may contain an overhang on either the 5′ or 3′ terminus producing an extension of single stranded character. The double stranded compounds optionally can include overhangs on the ends. Further modifications can include conjugate groups attached to one of the termini, selected nucleobase positions, sugar positions or to one of the internucleoside linkages. Alternatively, the two strands can be linked via a non-nucleic acid moiety or linker group. When formed from only one strand, dsRNA can take the form of a self-complementary hairpin-type molecule that doubles back on itself to form a duplex. Thus, the dsRNAs can be fully or partially double stranded. Specific modulation of gene expression can be achieved by stable expression of dsRNA hairpins in transgenic cell lines, however, in preferred embodiments, the gene expression is up regulated. When formed from two strands, or a single strand that takes the form of a self-complementary hairpin-type molecule doubled back on itself to form a duplex, the two strands (or duplex-forming regions of a single strand) are complementary RNA strands that base pair in Watson-Crick fashion.

In another embodiment, the desired oligonucleotides or antisense compounds, comprise at least one of: antisense RNA, antisense DNA, chimeric antisense oligonucleotides, antisense oligonucleotides comprising modified linkages, interference RNA (RNAi), short interfering RNA (siRNA); a micro, interfering RNA (miRNA); a small, temporal RNA (stRNA); or a short, hairpin RNA (shRNA); small RNA-induced gene activation (RNAa); small activating RNAs (saRNAs), or combinations thereof.

dsRNA can also activate gene expression, a mechanism that has been termed “small RNA-induced gene activation” or RNAa. dsRNAs targeting gene promoters induce potent transcriptional activation of associated genes. RNAa was demonstrated in human cells using synthetic dsRNAs, termed “small activating RNAs” (saRNAs).

Small double-stranded RNA (dsRNA) may also act as small activating RNAs (saRNA). Without wishing to be bound by theory, by targeting sequences in gene promoters, saRNAs would induce target gene expression in a phenomenon referred to as dsRNA-induced transcriptional activation (RNAa).

In some embodiments, the ribonucleic acid sequence is specific for regulatory segments of the genome that control the transcription of BAG3. Thus a candidate therapeutic agent can be a dsRNA that activates the expression of BAG3 in a cell and is administered to a patient in need of treatment.

Peptides: In another embodiment, a BAG3 peptide is encoded by a nucleic acid comprising a BCL2-associated athanogene 3 (BAG3) wild type, chimeric or cDNA sequences thereof. The peptide can also be a synthetic peptide of BCL2-associated athanogene 3 (BAG3).

It is to be understood that the peptide sequences are not limited to the native or cDNA sequences thereof, of BCL2-associated athanogene 3 (BAG3) molecules. The skilled artisan will recognize that conservative amino acid changes may be made, which although they alter the primary sequence of the protein or peptide, do not normally alter its function. Conservative amino acid substitutions typically include substitutions within the following groups: glycine, alanine, valine, isoleucine, leucine; aspartic acid, glutamic acid, asparagine, glutamine, serine, threonine, lysine, arginine, phenylalanine, tyrosine.

Embodiments of the invention also include polynucleotides encoding hybrid proteins comprising BCL2-associated athanogene 3 (BAG3) polypeptide operatively fused directly or indirectly via peptide linker, to a second polypeptide sequence. Linker sequences are well known in the art. Hybrid proteins comprising a BAG3 polypeptide or fragment thereof may be linked to other types of polypeptides. These additional polypeptides may be any amino acid sequence useful for the purification, identification, and/or therapeutic or prophylactic application of the peptide. In addition, the additional polypeptide can be a signal peptide, or targeting peptide, etc.

In some cases, the other additions, substitutions or deletions may increase the stability (including but not limited to, resistance to proteolytic degradation) of the polypeptide or increase affinity of the polypeptide for its appropriate receptor, ligand and/or binding proteins. In some cases, the other additions, substitutions or deletions may increase the solubility of the polypeptide. In some embodiments sites are selected for substitution with a naturally encoded or non-natural amino acid in addition to another site for incorporation of a non-natural amino acid for the purpose of increasing the polypeptide solubility following expression in recombinant host cells. In some embodiments, the polypeptides comprise another addition, substitution, or deletion that modulates affinity for the associated ligand, binding proteins, and/or receptor, modulates (including but not limited to, increases or decreases) receptor dimerization, stabilizes receptor dimers, modulates circulating half-life, modulates release or bio-availability, facilitates purification, or improves or alters a particular route of administration. Similarly, the non-natural amino acid polypeptide can comprise chemical or enzyme cleavage sequences, protease cleavage sequences, reactive groups, antibody-binding domains (including but not limited to, FLAG or poly-His) or other affinity based sequences (including but not limited to, FLAG, poly-His, GST, etc.) or linked molecules (including but not limited to, biotin) that improve detection (including but not limited to, GFP), purification, transport through tissues or cell membranes, prodrug release or activation, size reduction, or other traits of the polypeptide.

The methods and compositions described herein include incorporation of one or more non-natural amino acids into a polypeptide. One or more non-natural amino acids may be incorporated at one or more particular positions which does not disrupt activity of the polypeptide. This can be achieved by making “conservative” substitutions, including but not limited to, substituting hydrophobic amino acids with non-natural or natural hydrophobic amino acids, bulky amino acids with non-natural or natural bulky amino acids, hydrophilic amino acids with non-natural or natural hydrophilic amino acids) and/or inserting the non-natural amino acid in a location that is not required for activity.

Gene-Editing Agents

In certain embodiments, the agent comprises one or more gene-editing or nuclease systems to delete or edit HIV genes in a subject, e.g. Tat. The target nucleic acid can be in any gene of the virus, as long as Tat expression of functions are inhibited.

Any suitable nuclease system can be used including, for example, clustered regularly interspaced short palindromic repeat (CRISPR) nucleases, Argonaute family of endonucleases, zinc-finger nucleases (ZFNs), transcription activator-like effector nucleases (TALENs), meganucleases, other endo- or exo-nucleases, or combinations thereof. See Schiffer, 2012, J Virol 88(17):8920-8936, incorporated by reference.

Candidate Agents and Screening Assays

The compositions embodied herein, can also be applied in the areas of drug discovery and target validation. The present invention comprehends the use of the nucleic acid sequences and peptides embodied herein, in drug discovery efforts to elucidate relationships that exist between Bcl-2 associated anthanogene-3 (BAG3) polynucleotides and HIV Tat molecules in a disease state, phenotype, or condition.

The screening assays of the invention suitably include and embody, animal models, cell-based systems and non-cell based systems. The nucleic acid sequences and peptides embodied herein, are used for identifying agents of therapeutic interest, e.g. by screening libraries of compounds or otherwise identifying compounds of interest by any of a variety of drug screening or analysis techniques, or synthesis of novel compounds. The gene, allele, fragment, or oligopeptide thereof employed in such screening may be free in solution, affixed to a solid support, borne on a cell surface, or located intracellularly. The measurements are conducted as described in detail in the examples section which follows, e.g. accumulation of hyperphosphorylated tau, mitochondrial dysfunction, co-localization of BAG3 and HSP70, mitophagy, ATP generation, etc. In embodiments, screening candidate agents is performed to identify those which modulate the interactions between BAG3, Tat and/or hsp70.

The assays can be of an in vitro or in vivo format. In vitro formats of interest include cell-based formats, in which contact occurs e.g., by introducing the substrate in a medium, such as an aqueous medium, in which the cell is present. In yet other embodiments, the assay may be in vivo, in which a multicellular organism that includes the cell is employed. Contact of a targeting vector encoding the nucleic acid sequences embodied herein, with the target cell(s) may be accomplished using any convenient protocol. In those embodiments where the target cells are present as part of a multicellular organism, e.g., an animal, the vector is conveniently administered to (e.g., injected into, fed to, etc.) the multicellular organism, e.g., a whole animal, where administration may be systemic or localized, e.g., directly to specific tissue(s) and/or organ(s) of the multicellular organism.

Multicellular organisms of interest include, but are not limited to: insects, vertebrates, such as avian species, e.g., chickens; mammals, including rodents, e.g., mice, rates; ungulates, e.g., pigs, cows, horses; dogs, cats, primates, e.g., monkeys, apes, humans; and the like. As such, the target cells of interest include, but are not limited to: insects cells, vertebrate cells, particularly avian cells, e.g., chicken cells; mammalian cells, including murine, porcine, ungulate, ovine, equine, rat, dog, cat, monkey, and human cells; and the like.

In certain embodiments, the subject methods are performed in a high throughput (HT) format. In the subject HT embodiments of the subject invention, a plurality of different cells are simultaneously assayed or tested. By simultaneously tested is meant that each of the cells in the plurality are tested at substantially the same time. In general, the number of cells that are tested simultaneously in the subject HT methods ranges from about 10 to 10,000, usually from about 100 to 10,000 and in certain embodiments from about 1000 to 5000. A variety of high throughput screening assays for determining the activity of candidate agent are known in the art and are readily adapted to the present invention, including those described in e.g., Schultz (1998) Bioorg Med Chem Lett 8:2409-2414; Fernandes (1998) Curr Opin Chem Biol 2:597-603; as well as those described in U.S. Pat. No. 6,127,133; the disclosures of which are herein incorporated by reference.

Candidate Agents: The methods can be practiced with any test compounds as candidate agents. Test compounds useful in practicing the inventive method may be obtained using any of the numerous approaches in combinatorial library methods known in the art, including biological libraries, spatially-addressable parallel solid phase or solution phase libraries, synthetic library methods requiring deconvolution, the “one-bead one-compound” library method, and synthetic library methods using affinity chromatography selection. The biological library approach is limited to peptide libraries, while the other four approaches are applicable to peptide, nonpeptide oligomer, or small molecule libraries of compounds (Lam, 1997, Anticancer Drug Des. 12:145).

In certain embodiments, a candidate agent comprises antibodies, antibody fragments, small molecules, oligonucleotides, polynucleotides, peptides, polypeptides, proteins, gene-editing agents, siRNA, enzymes, peptide nucleic acids, organic molecules, synthetic molecules or combinations thereof.

The method may be practiced iteratively using different concentrations of a test candidate and/or different testing conditions, such as duration of reaction time. Test candidates that are identified by the method can be further tested by conventional methods in the art to verify specificity, dose dependency, efficacy in vivo, and the like. Test candidates may serve as lead compounds for developing additional test candidates.

A prototype compound or agent may be believed to have therapeutic activity on the basis of any information available to the artisan. For example, a prototype agent may be believed to have therapeutic activity on the basis of information contained in the Physician's Desk Reference. In addition, by way of non-limiting example, a compound may be believed to have therapeutic activity on the basis of experience of a clinician, structure of the compound, structural activity relationship data, EC₅₀, assay data, IC₅₀ assay data, animal or clinical studies, or any other basis, or combination of such bases.

A therapeutically-active compound or agent is an agent that has therapeutic activity, including for example, the ability of the agent to induce a specified response when administered to a subject or tested in vitro. Therapeutic activity includes treatment of a disease or condition, including both prophylactic and ameliorative treatment. Treatment of a disease or condition can include improvement of a disease or condition by any amount, including prevention, amelioration, and elimination of the disease or condition. Therapeutic activity may be conducted against any disease or condition, including in a preferred embodiment against an HIV infection or disorder associated with Tat-BAG3 interactions. In order to determine therapeutic activity any method by which therapeutic activity of a compound may be evaluated can be used. For example, both in vivo and in vitro methods can be used, including for example, clinical evaluation, EC₅₀, and IC₅₀ assays, and dose response curves.

Candidate compounds for use with an assay of the present invention or identified by assays of the present invention as useful pharmacological agents can be pharmacological agents already known in the art or variations thereof or can be compounds previously unknown to have any pharmacological activity. The candidate compounds can be naturally occurring or designed in the laboratory. Candidate compounds can comprise a single diastereomer, more than one diastereomer, or a single enantiomer, or more than one enantiomer.

Candidate compounds can be isolated, from microorganisms, animals or plants, for example, and can be produced recombinantly, or synthesized by chemical methods known in the art. If desired, candidate compounds of the present invention can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection. The biological library approach is limited to polypeptide libraries. The other four approaches are applicable to polypeptide, non-peptide oligomers, or small molecule libraries of compounds and are preferred approaches in the present invention. See Lam, Anticancer Drug Des. 12: 145-167 (1997).

In an embodiment, the present invention provides a method of identifying a candidate compound as a suitable prodrug. A suitable prodrug includes any prodrug that may be identified by the methods of the present invention. Any method apparent to the artisan may be used to identify a candidate compound as a suitable prodrug.

In another aspect, the present invention provides methods of screening candidate compounds for suitability as therapeutic agents. Screening for suitability of therapeutic agents may include assessment of one, some or many criteria relating to the compound that may affect the ability of the compound as a therapeutic agent. Factors such as, for example, efficacy, safety, efficiency, retention, localization, tissue selectivity, degradation, or intracellular persistence may be considered. In an embodiment, a method of screening candidate compounds for suitability as therapeutic agents is provided, where the method comprises providing a candidate compound identified as a suitable prodrug, determining the therapeutic activity of the candidate compound, and determining the intracellular persistence of the candidate compound. Intracellular persistence can be measured by any technique apparent to the skilled artisan, such as for example by radioactive tracer, heavy isotope labeling, or LCMS.

In screening compounds for suitability as therapeutic agents, intracellular persistence of the candidate compound is evaluated. In a preferred embodiment, the agents are evaluated for their ability to modulate the translation of compositions embodied herein, over a period of time in response to a candidate therapeutic agent.

In another embodiment, soluble and/or membrane-bound forms of compositions embodied herein, e.g. proteins, mutants or biologically active portions thereof, can be used in the assays for screening candidate agents. When membrane-bound forms of the protein are used, it may be desirable to utilize a solubilizing agent. Examples of such solubilizing agents include non-ionic detergents such as n-octylglucoside, n-dodecylglucoside, n-dodecylmaltoside, octanoyl-N-methylglucamide, decanoyl-N-methylglucamide, TRITON™ X-100, TRITON™ X-114, THESIT™, Isotridecypoly(ethylene glycol ether)n, 3-[(3-cholamidopropyl)dimethylamminio]-1-propane sulfonate (CHAPS), 3-[(3-cholamidopropyl)dimethylamminio]-2-hydroxy-1-propane sulfonate (CHAPSO), or N-dodecyl=N,N-dimethyl-3-ammonio-1-propane sulfonate.

Cell-free assays can also be used and involve preparing a reaction mixture which includes BAG3 molecules (nucleic acids or peptides) and Tat comprising a bioluminescent moiety and the test compound under conditions and time periods to allow the measurement of the BAG3/Tat interactions as described in the examples section which follows.

Combination Therapies

In certain embodiments, the agents and pharmaceutical compositions thereof embodied herein, are administered to a patient in combination with one or more other anti-viral agents or therapeutics. Examples include any molecules that are used for the treatment of a virus and include agents which alleviate any symptoms associated with the virus, for example, anti-pyretic agents, anti-inflammatory agents, chemotherapeutic agents, and the like. An antiviral agent includes, without limitation: antibodies, aptamers, adjuvants, anti-sense oligonucleotides, chemokines, cytokines, immune stimulating agents, immune modulating agents, B-cell modulators, T-cell modulators, NK cell modulators, antigen presenting cell modulators, enzymes, siRNA's, ribavirin, protease inhibitors, helicase inhibitors, polymerase inhibitors, helicase inhibitors, neuraminidase inhibitors, nucleoside reverse transcriptase inhibitors, non-nucleoside reverse transcriptase inhibitors, purine nucleosides, chemokine receptor antagonists, interleukins, or combinations thereof.

In certain embodiments, the agents and pharmaceutical compositions thereof embodied herein are administered with one or more compositions comprising a therapeutically effective amount of a non-nucleoside reverse transcriptase inhibitor (NNRTI) and/or a nucleoside reverse transcriptase inhibitor (NRTI), analogs, variants or combinations thereof. In certain embodiments, an NNRTI comprises: etravirine, efavirenz, nevirapine, rilpivirine, delavirdine, or nevirapine. In embodiments, an NRTI comprises: lamivudine, zidovudine, emtricitabine, abacavir, zalcitabine, dideoxycytidine, azidothymidine, tenofovir disoproxil fumarate, didanosine (ddl EC), dideoxyinosine, stavudine, abacavir sulfate or combinations thereof. In certain embodiments, a composition comprises a therapeutically effective amount of at least one NNRTI or a combination of NNRTI's, analogs, variants or combinations thereof. In certain embodiments, the NNRTI is rilpivirine. In certain embodiments, an NRTI comprises: lamivudine, zidovudine, emtricitabine, abacavir, zalcitabine, dideoxycytidine, azidothymidine, tenofovir disoproxil fumarate, didanosine (ddl EC), dideoxyinosine, stavudine, abacavir sulfate or combinations thereof. In certain embodiments, the composition comprises a therapeutically effective amount of at least one or a combination of NRTI's, analogs, variants or combinations thereof.

Methods of Treatment

In certain embodiments, a method of preventing or treating neurodegeneration in a subject infected with human immunodeficiency virus, comprising: administering to the subject a pharmaceutical composition comprising a therapeutically effective amount of an agent which inhibits interaction between human immunodeficiency virus (HIV) Tat protein, BAG-3 and/or heat shock protein 70 (HSP70), thereby preventing or treating dementia in the subject.

In certain embodiments, a method of treating preventing or treating neurodegeneration in a subject infected with HIV, comprises administering Bcl2-associated anthanogene 3 (BAG3) polynucleotide, polypeptide and/or agent which induce BAG3 expression or function.

In certain embodiments, a method of preventing and/or treating dementia in a subject infected with HIV, comprises administering Bcl2-associated anthanogene 3 (BAG3) polynucleotide, polypeptide and/or agent which induce BAG3 expression or function.

In certain embodiments, an agent decreases expression or function of Tat.

In methods of treatment of neurodegenerative diseases, e.g. dementia, in a subject having an HIV infection, a subject can be identified using standard clinical tests, for example, immunoassays to detect the presence of HIV antibodies or the HIV polypeptide p24 in the subject's serum, or through HIV nucleic acid amplification assays. An amount of such a composition provided to the subject that results in a complete resolution of the symptoms of the infection, a decrease in the severity of the symptoms of the infection, or a slowing of the infection's progression is considered a therapeutically effective amount. The present methods may also include a monitoring step to help optimize dosing and scheduling as well as predict outcome. In some methods of the present invention, one can first determine whether a patient has a latent HIV infection, and then make a determination as to whether or not to treat the patient with one or more of the compositions described herein.

Pharmaceutical Compositions

Pharmaceutical compositions according to the present invention can be prepared in a variety of ways known to one of ordinary skill in the art. For example, the agents described above can be formulated in compositions for application to cells in tissue culture or for administration to a patient or subject. These compositions can be prepared in a manner well known in the pharmaceutical art, and can be administered by a variety of routes, depending upon whether local or systemic treatment is desired and upon the area to be treated. Administration may be topical (including ophthalmic and to mucous membranes including intranasal, vaginal and rectal delivery), pulmonary (e.g., by inhalation or insufflation of powders or aerosols, including by nebulizer; intratracheal, intranasal, epidermal and transdermal), ocular, oral or parenteral. Methods for ocular delivery can include topical administration (eye drops), subconjunctival, periocular or intravitreal injection or introduction by balloon catheter or ophthalmic inserts surgically placed in the conjunctival sac. Parenteral administration includes intravenous, intraarterial, subcutaneous, intraperitoneal or intramuscular injection or infusion; or intracranial, e.g., intrathecal or intraventricular administration. Parenteral administration can be in the form of a single bolus dose, or may be, for example, by a continuous perfusion pump. Pharmaceutical compositions and formulations for topical administration may include transdermal patches, ointments, lotions, creams, gels, drops, suppositories, sprays, liquids, powders, and the like. Conventional pharmaceutical carriers, aqueous, powder or oily bases, thickeners and the like may be necessary or desirable.

This invention also includes pharmaceutical compositions which contain, as the active ingredient, agents described herein, in combination with one or more pharmaceutically acceptable carriers. The terms “pharmaceutically acceptable” (or “pharmacologically acceptable”) refer to molecular entities and compositions that do not produce an adverse, allergic or other untoward reaction when administered to an animal or a human, as appropriate. The term “pharmaceutically acceptable carrier,” as used herein, includes any and all solvents, dispersion media, coatings, antibacterial, isotonic and absorption delaying agents, buffers, excipients, binders, lubricants, gels, surfactants and the like, that may be used as media for a pharmaceutically acceptable substance. In making the compositions of the invention, the active ingredient is typically mixed with an excipient, diluted by an excipient or enclosed within such a carrier in the form of, for example, a capsule, tablet, sachet, paper, or other container. When the excipient serves as a diluent, it can be a solid, semisolid, or liquid material (e.g., normal saline), which acts as a vehicle, carrier or medium for the active ingredient. Thus, the compositions can be in the form of tablets, pills, powders, lozenges, sachets, cachets, elixirs, suspensions, emulsions, solutions, syrups, aerosols (as a solid or in a liquid medium), lotions, creams, ointments, gels, soft and hard gelatin capsules, suppositories, sterile injectable solutions, and sterile packaged powders. As is known in the art, the type of diluent can vary depending upon the intended route of administration. The resulting compositions can include additional agents, such as preservatives. In some embodiments, the carrier can be, or can include, a lipid-based or polymer-based colloid. In some embodiments, the carrier material can be a colloid formulated as a liposome, a hydrogel, a microparticle, a nanoparticle, or a block copolymer micelle. As noted, the carrier material can form a capsule, and that material may be a polymer-based colloid.

In some embodiments the composition is administered utilizing a polymeric, biodegradable microparticle or microcapsule delivery vehicle, sized to optimize phagocytosis by phagocytic cells such as macrophages. For example, PLGA (poly-lacto-co-glycolide) microparticles approximately 1-10 μm in diameter can be used. The agent is encapsulated in these microparticles, which are taken up by macrophages and gradually biodegraded within the cell, thereby releasing the polynucleotide. A second type of microparticle is intended not to be taken up directly by cells, but rather to serve primarily as a slow-release reservoir of nucleic acid that is taken up by cells only upon release from the micro-particle through biodegradation. These polymeric particles should therefore be large enough to preclude phagocytosis (i.e., larger than 5 μm and preferably larger than 20 μm). Another way to achieve uptake of the agent is using liposomes, prepared by standard methods. The agents can be incorporated alone into these delivery vehicles or co-incorporated with tissue-specific antibodies, for example antibodies that target cell types that are common latently infected reservoirs of HIV infection, for example, brain macrophages, microglia, astrocytes, and gut-associated lymphoid cells. Ain some embodiments, the compositions of the invention can be formulated as a nanoparticle, for example, nanoparticles comprised of a core of high molecular weight linear polyethylenimine (LPEI) complexed with DNA and surrounded by a shell of polyethyleneglycol-modified (PEGylated) low molecular weight LPEI.

The excipient or carrier is selected on the basis of the mode and route of administration. Suitable pharmaceutical carriers, as well as pharmaceutical necessities for use in pharmaceutical formulations, are described in Remington's Pharmaceutical Sciences (E. W. Martin), a well-known reference text in this field, and in the USP/NF (United States Pharmacopeia and the National Formulary).

The invention has been described in detail with reference to preferred embodiments thereof. However, it will be appreciated that those skilled in the art, upon consideration of this disclosure, may make modifications and improvements within the spirit and scope of the invention.

All documents mentioned herein are incorporated herein by reference. All publications and patent documents cited in this application are incorporated by reference for all purposes to the same extent as if each individual publication or patent document were so individually denoted. By their citation of various references in this document, Applicants do not admit any particular reference is “prior art” to their invention.

### EXAMPLES

### Example 1: BAG Family and Tat Interactions

The link between the HIV Tat protein, the accumulation of aberrant and hp-tau and the common mechanisms by which they induce cell stress resulting in mitochondrial dysfunction via BAG3 and HSP70 were studied. Such studies bridge the gap between molecular mechanisms at the cellular level and neuropathogenesis to define the underlying pathophysiology of neuronal cell damage caused by tau accumulation. The end results will lead to new therapeutic strategies for the treatment and prevention of dementia in aging HIV⁺ patients.

In a first set of experiments, the levels of total tau and hyperphosphorylated tau (hp-tau) were examined in lysates from archival human brain tissue from patients with AD and HIVE. Total tau levels were similar in all cases, but hyperphosphorylated tau (hp-tau) was increased in AD as well as HIVE compared with normal brain tissues. Tissues were obtained from the Alzheimer's Disease Research Center (La Jolla, Calif.), California NeuroAIDS Tissue Consortium (San Diego, Calif.), National NeuroAIDS Tissue Consortium, University of Pennsylvania Center for Neurodegenerative Disease Research, Alzheimer's Disease Core Center (ADCC) and Udall Center for Parkinson's Research. FIG. 1A shows results from representative Western blot showing soluble hp-tau (s262) and total tau (HT7) in age-matched controls (normal), AD Braak stages 1, 3, 5 (BrI, 3, 5), and HIV encephalitis (HIVE) patients. FIG. 1B is a graphic representation of percent total tau that is hyperphosphorylated (hp-tau/total tau) in each case compared to control.

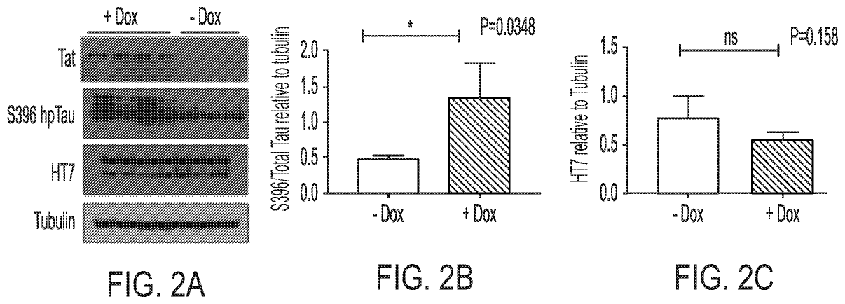

Results from experiments that the induction of Tat in the Tat transgenic mouse model induced increased levels of hp-tau (FIGS. 2A-2C). Doxycycline (Dox)-inducible GFAP promoter driven HIV-1 Tat transgenic mice were used. Mice were injected i.p. with doxycycline hyclate (+Dox) (Sigma-Aldrich) for 7 days at a dosage of 80 mg/kg/day to induce Tat. The control group (−Dox) received saline injections.

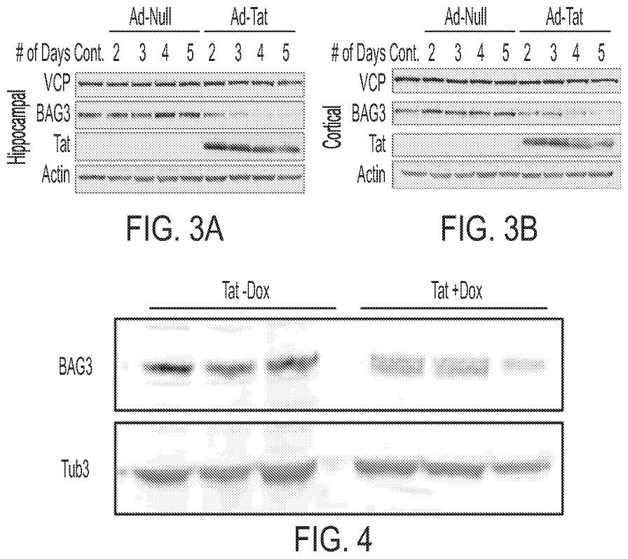

FIGS. 3A and 3B show the results from experiments conducted demonstrating suppression of BAG3 by Tat in neuronal cells. These results link Tat to regulation of protein quality control and autophage mediated by BAG3 in neuronal cells. The suppression of BAG3 was also demonstrated in transgenic animals expressing Tat. Similar to the results seen above in neuronal cells in culture (FIGS. 3A, 3B), levels of BAG3 in tissue from doxycycline (Dox)-inducible HIV-1 Tat transgenic mice were examined as described in FIGS. 2A-2C. Induction of Tat resulted in downregulation of BAG3 in vivo in lysates prepared from brain tissues of mice treated with dox (+Dox) versus saline injected control mice (−Dox). Thus, the in vitro and in vivo systems utilized for these studies demonstrate Tat-mediated control of PQC and mitophagy pathways via BAG3.

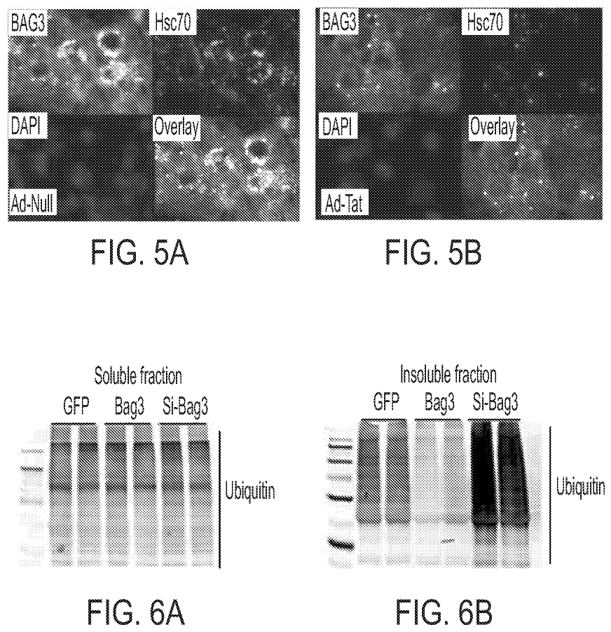

Tat disruption of co-localization of BAG3 and HSP70 in neuronal cells is shown in FIGS. 5A, 5B. Immunocytochemistry shows in the absence of Tat (Ad-null), BAG3 and HSP70/Hsc70 are found in the cytoplasm and co-localize predominantly in puncta seen in the perinuclear region. In the presence of Tat (Ad-Tat), puncta are substantially reduced and co-localization of BAG3 and HSP70/Hsc70 is reduced by the presence of Tat.

It was shown that while no differences in ubiquitinated proteins are detected in the soluble fractions of neuronal cells, overexpression of BAG3 (Bag3) decreased the level of ubiquitinated proteins in insoluble fractions. Conversely, silencing of BAG3 by siRNA (si-Bag3) profoundly increased the level of ubiquitinated proteins in the insoluble fraction, suggesting modulation of PQC by functional BAG3 in the cells. This proof-of-concept data shows the ability of BAG3 to control protein ubiquitination, a step toward degradation of protein by PQC (FIGS. 6A, 6B).

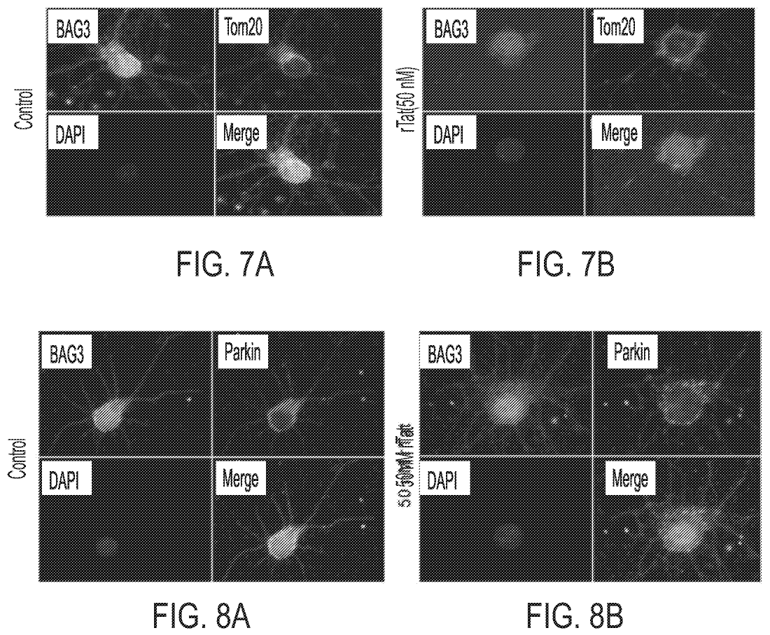

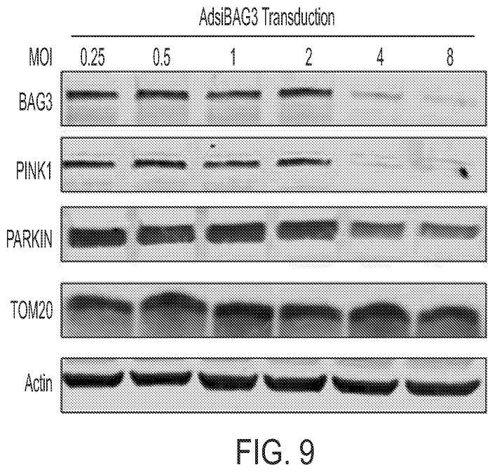

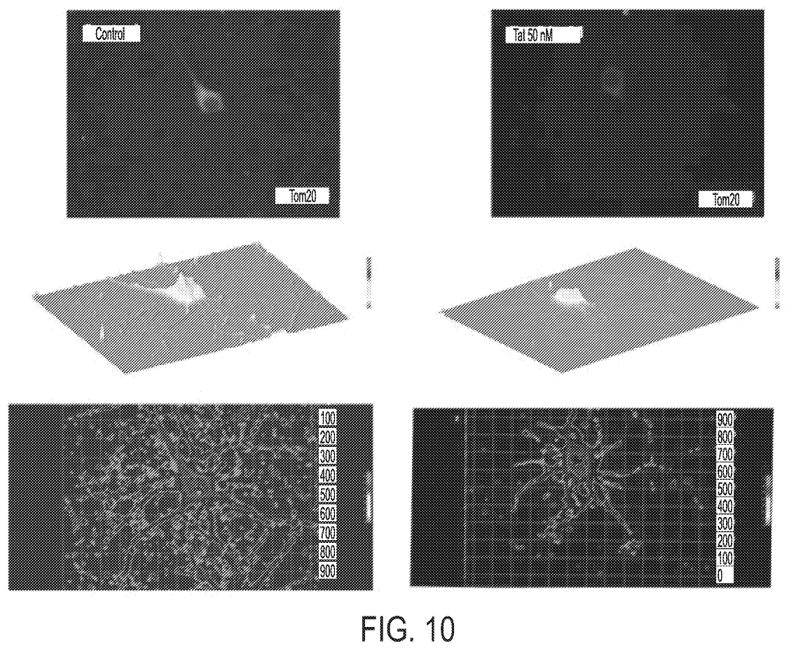

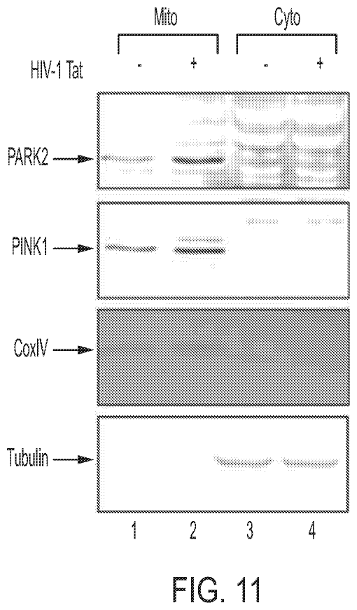

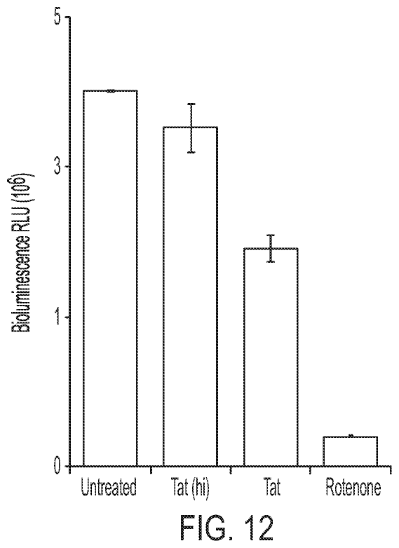

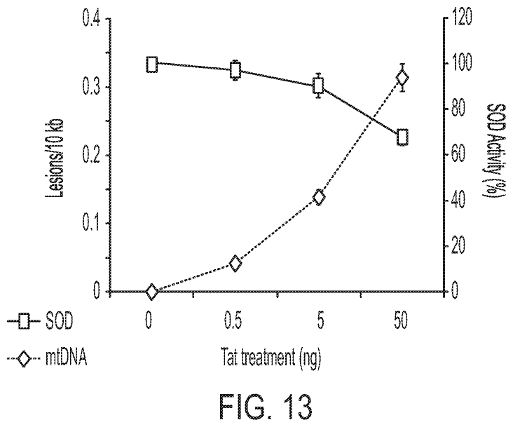

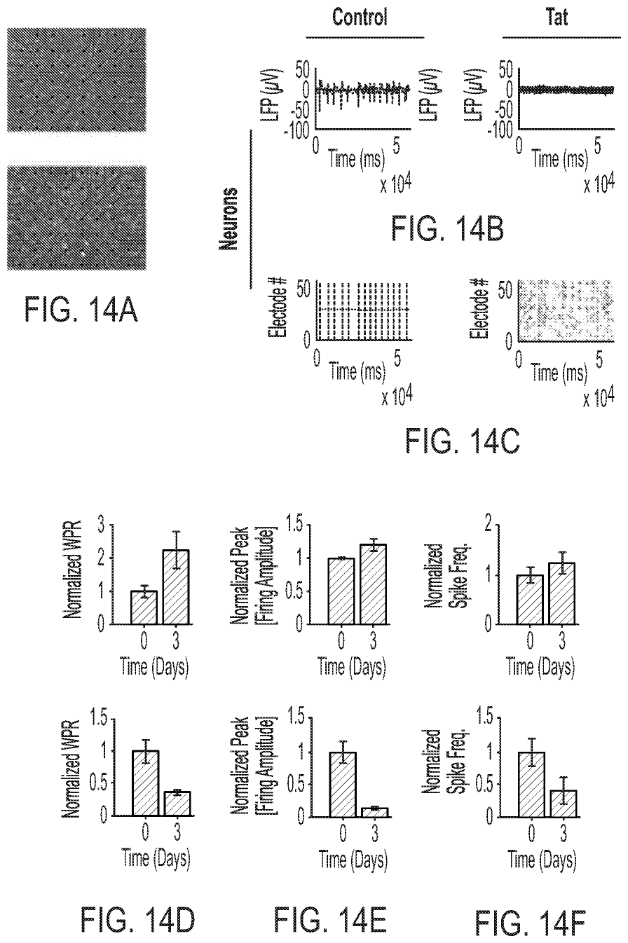

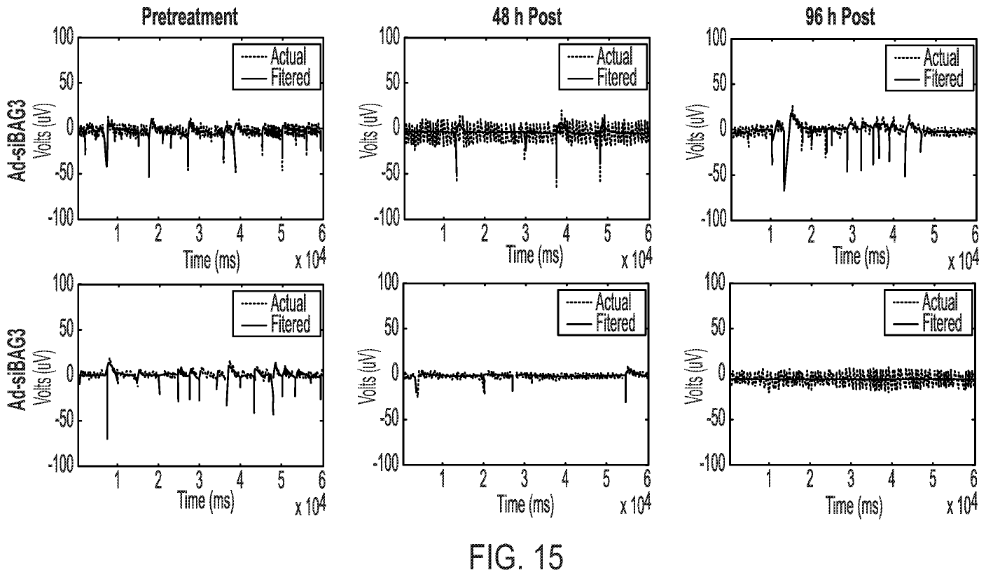

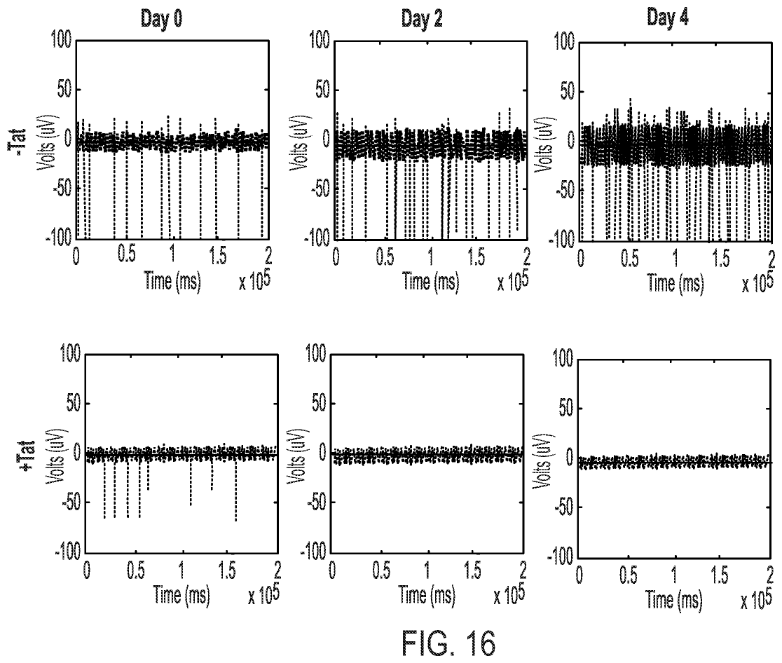

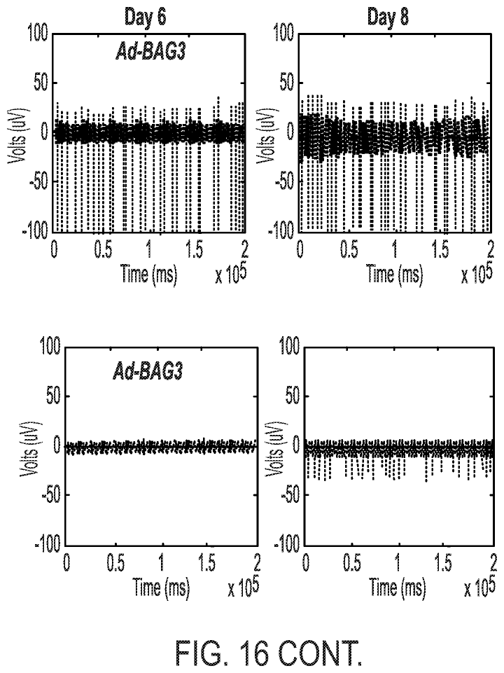

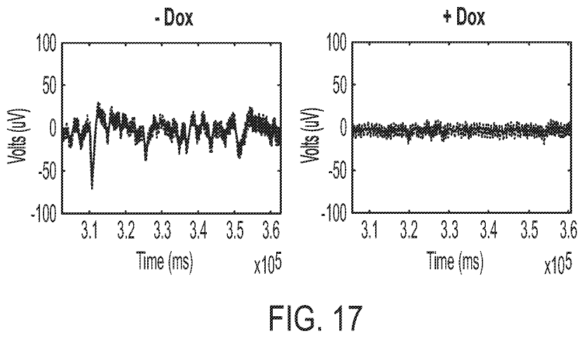

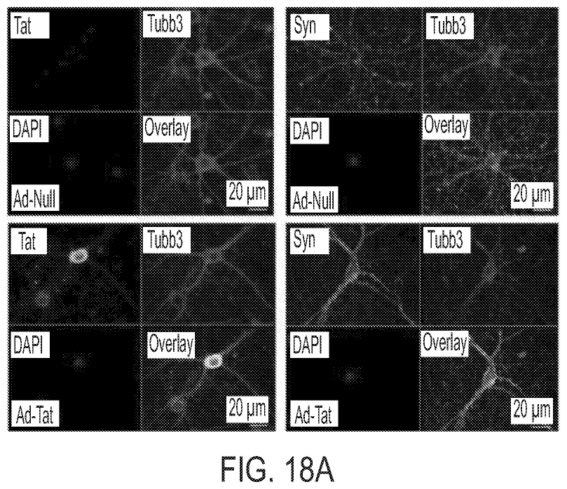

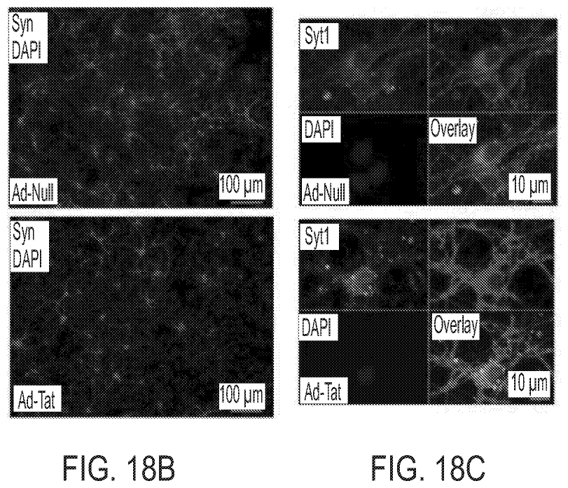

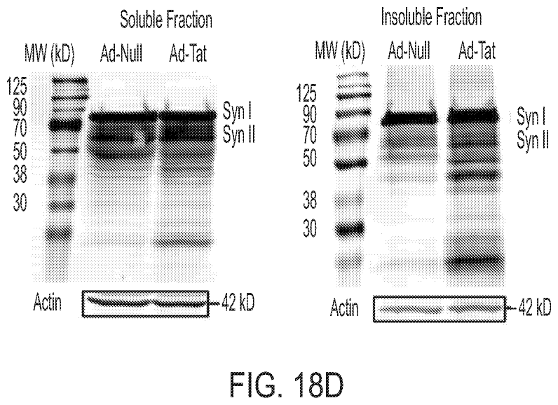

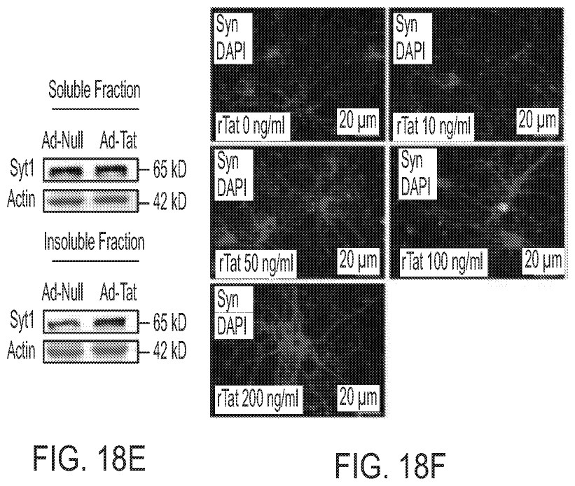

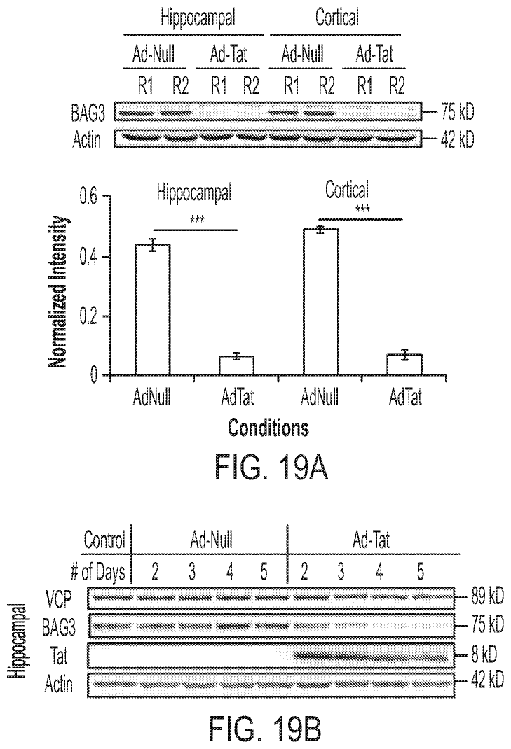

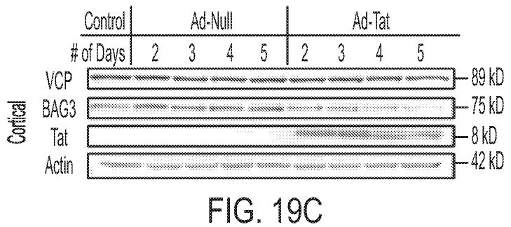

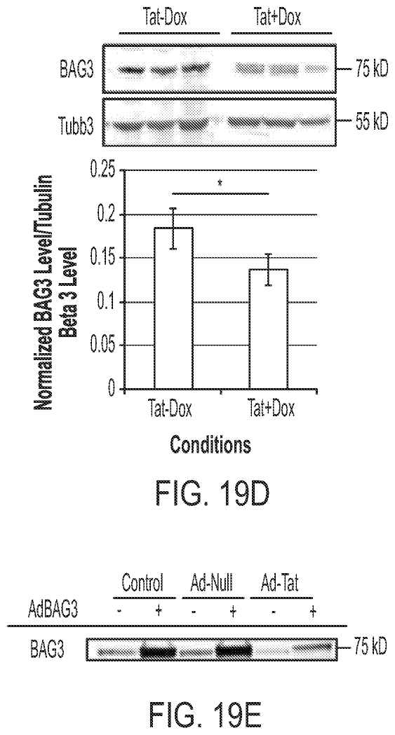

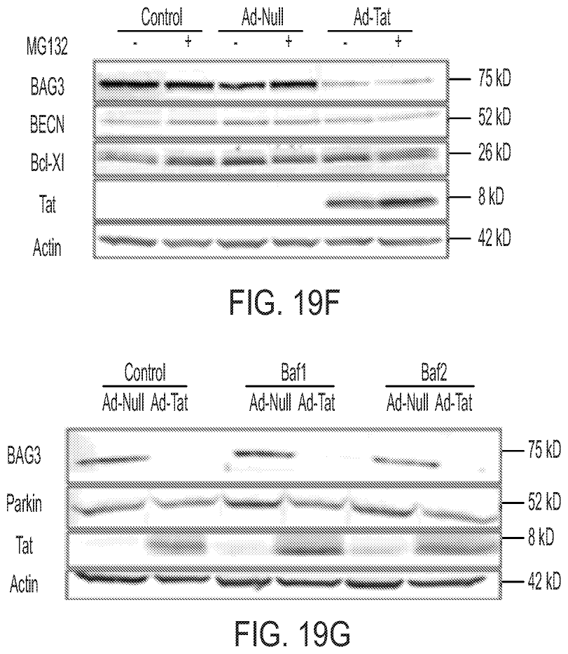

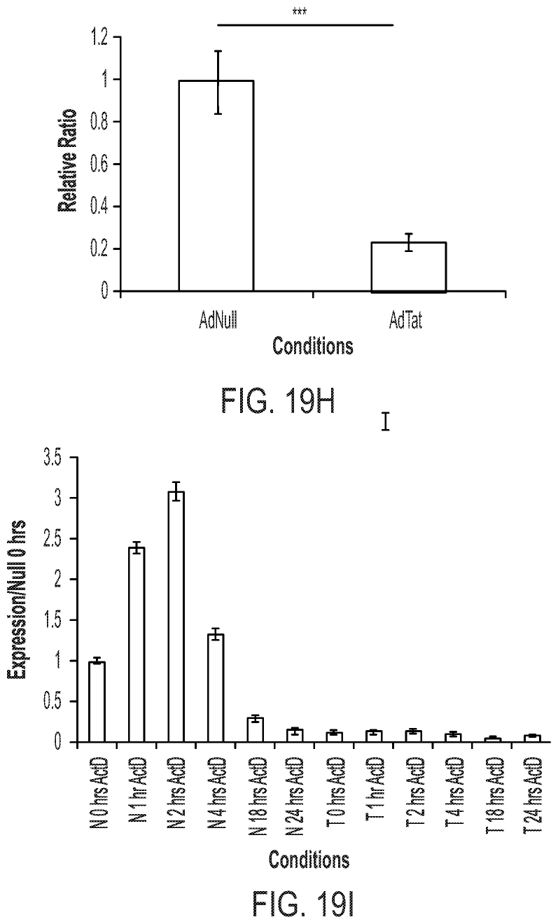

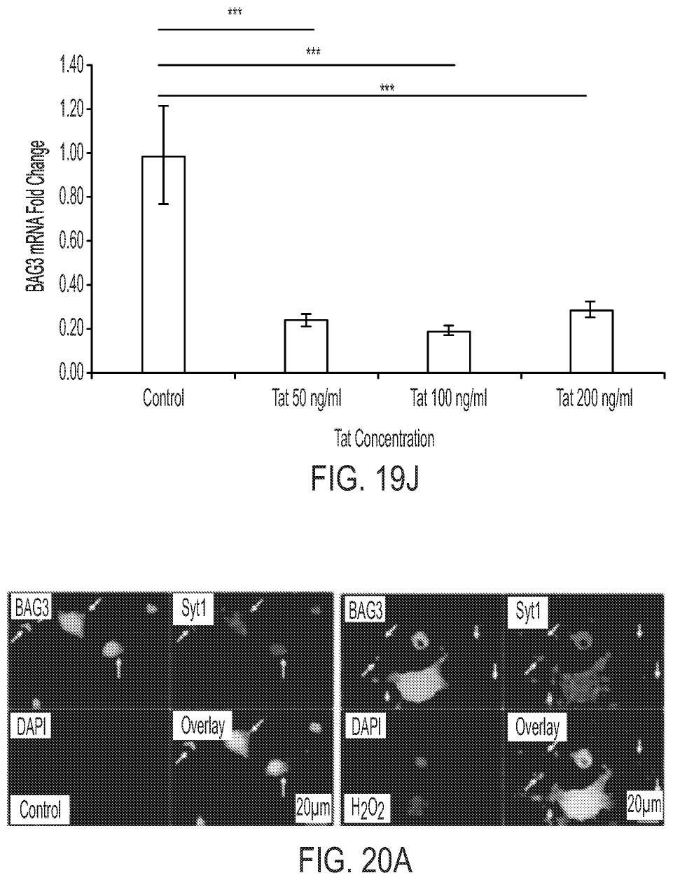

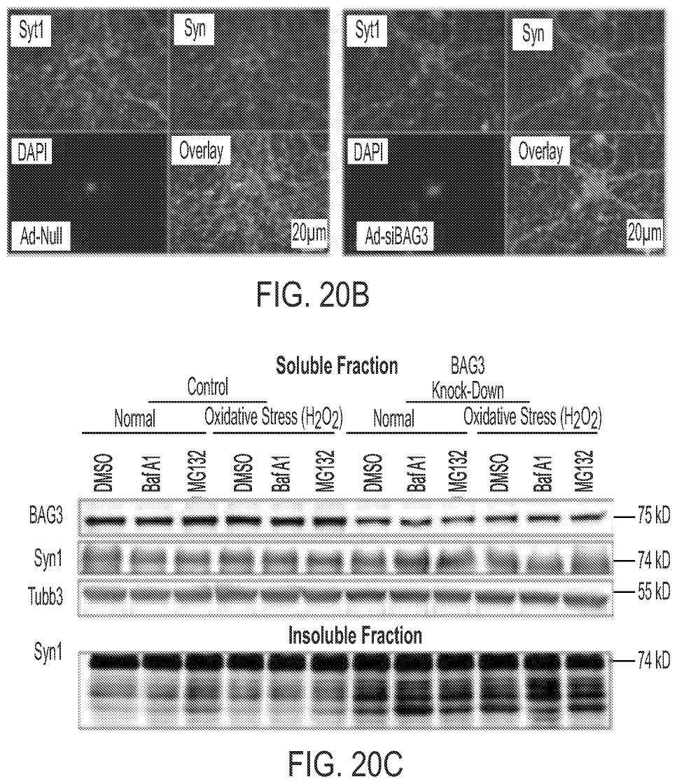

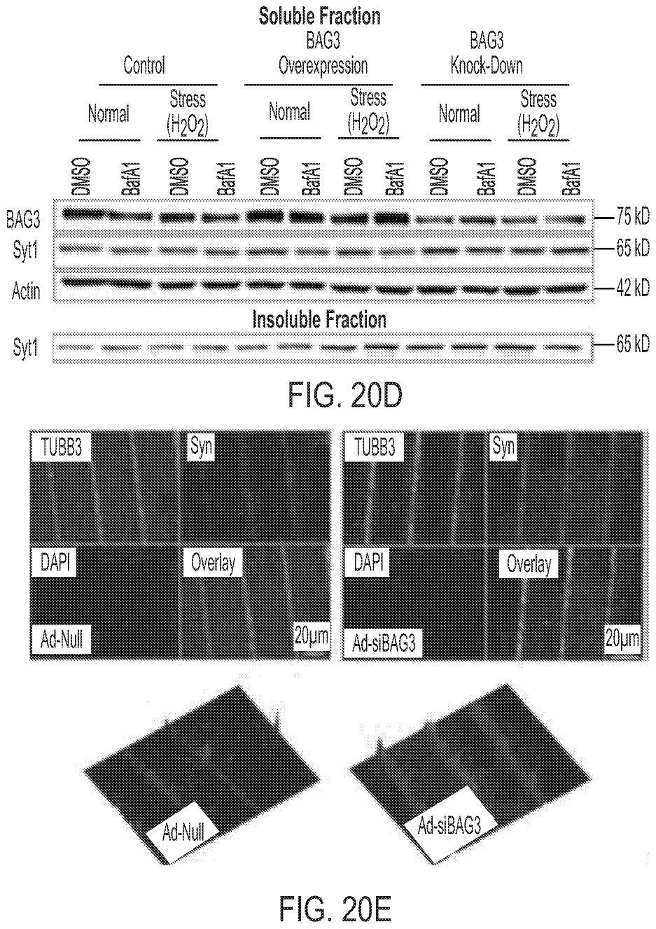

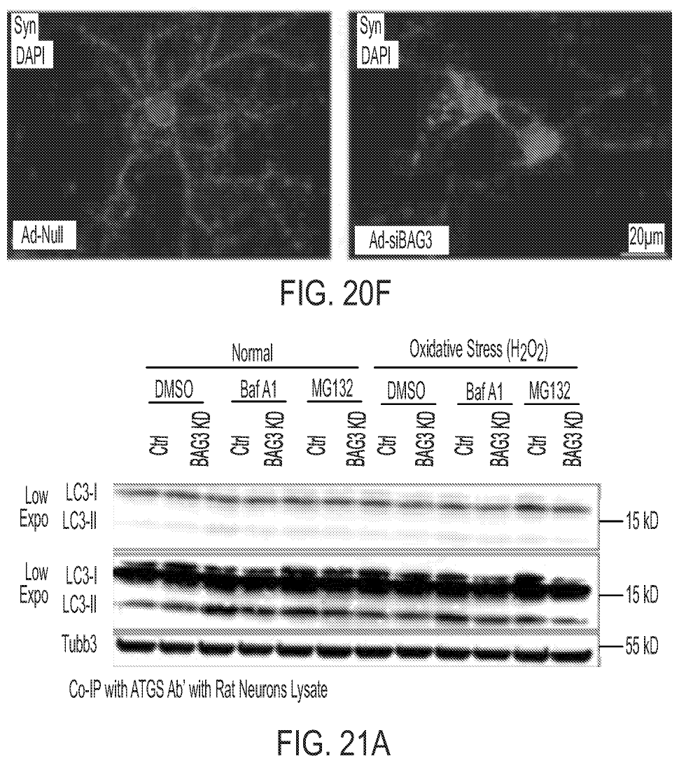

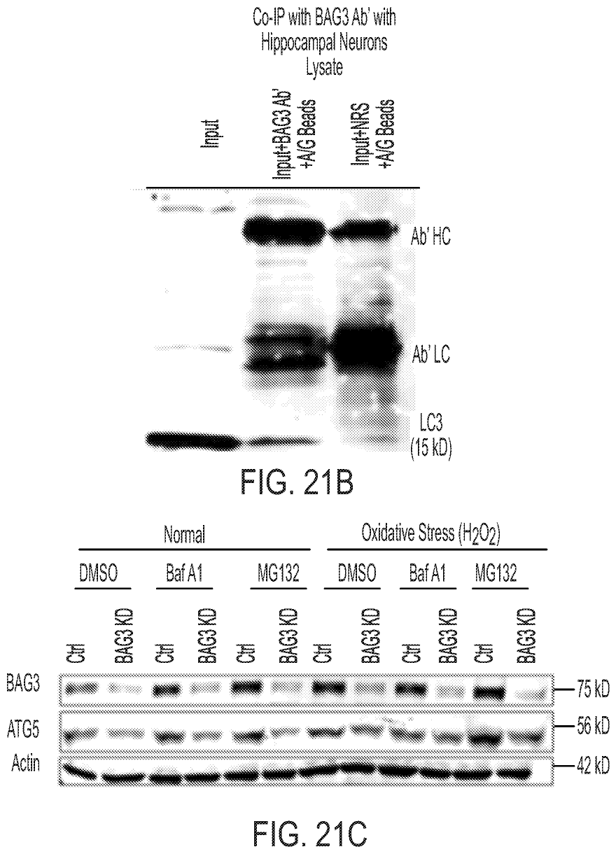

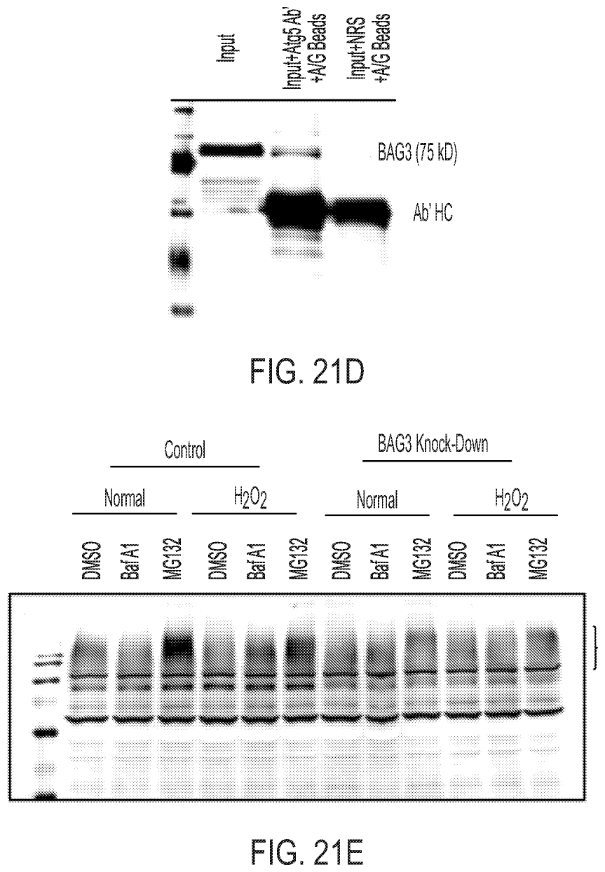

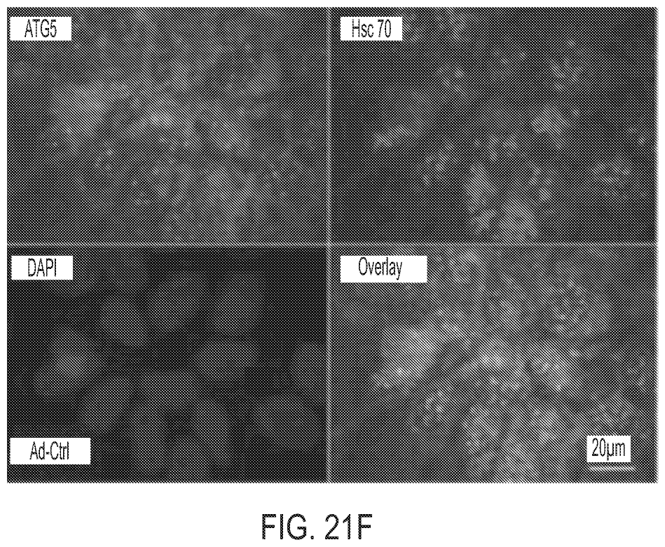

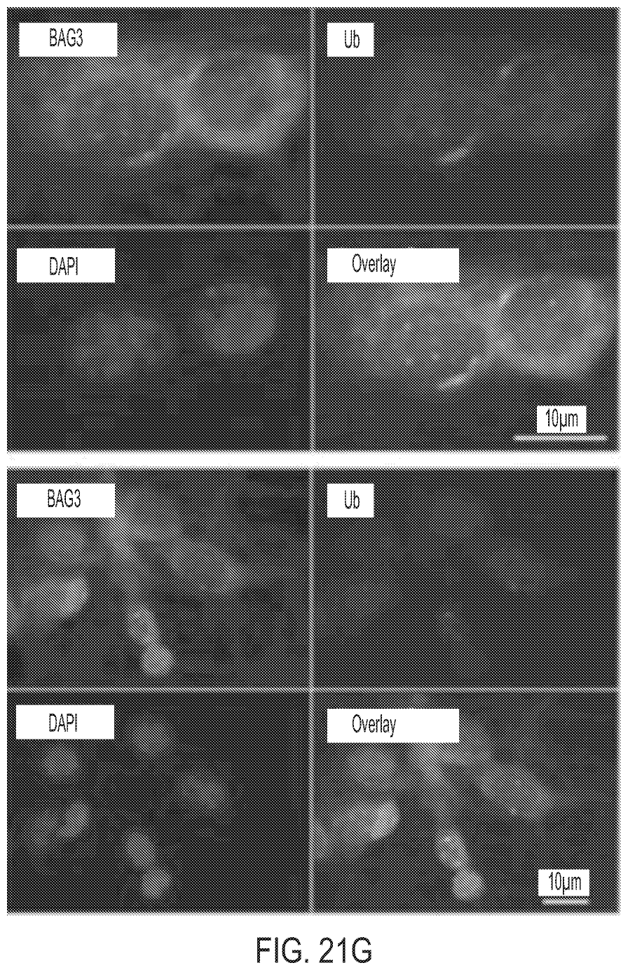

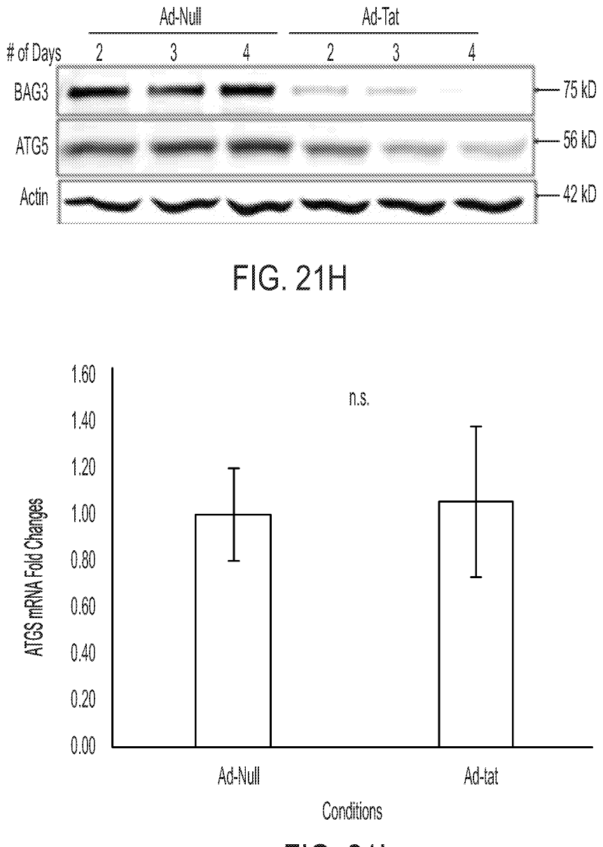

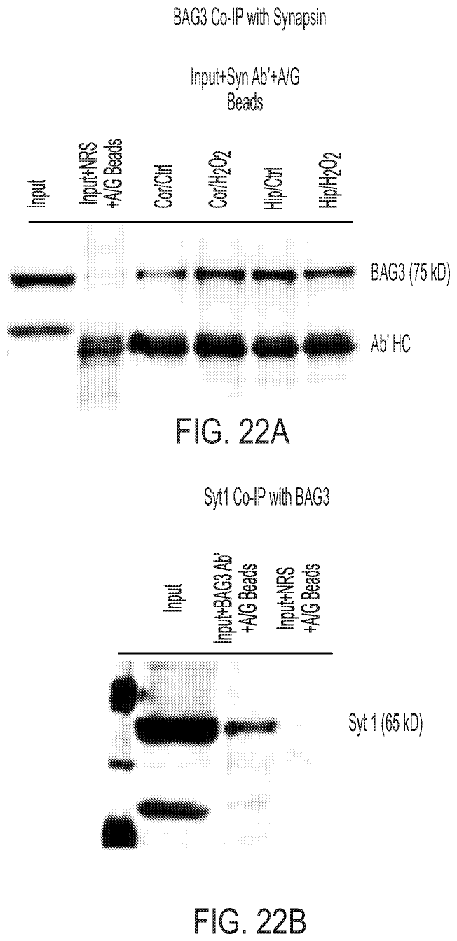

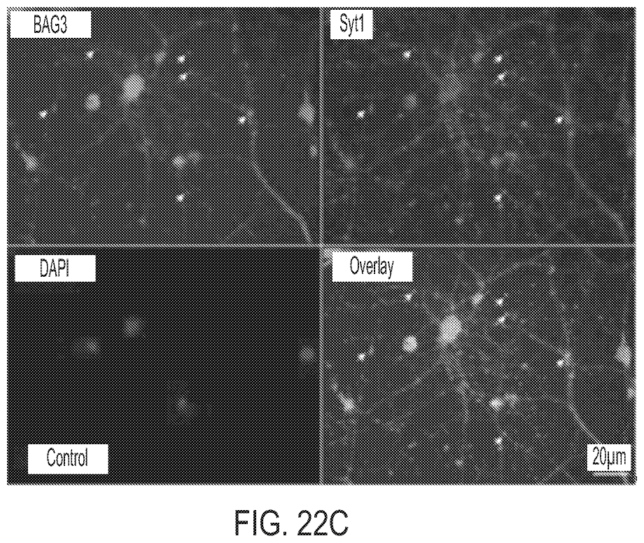

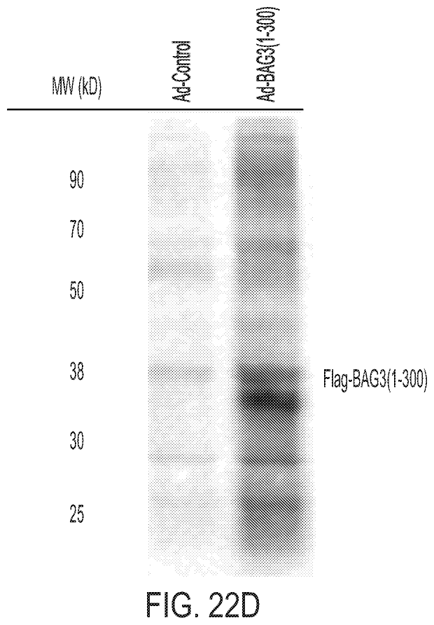

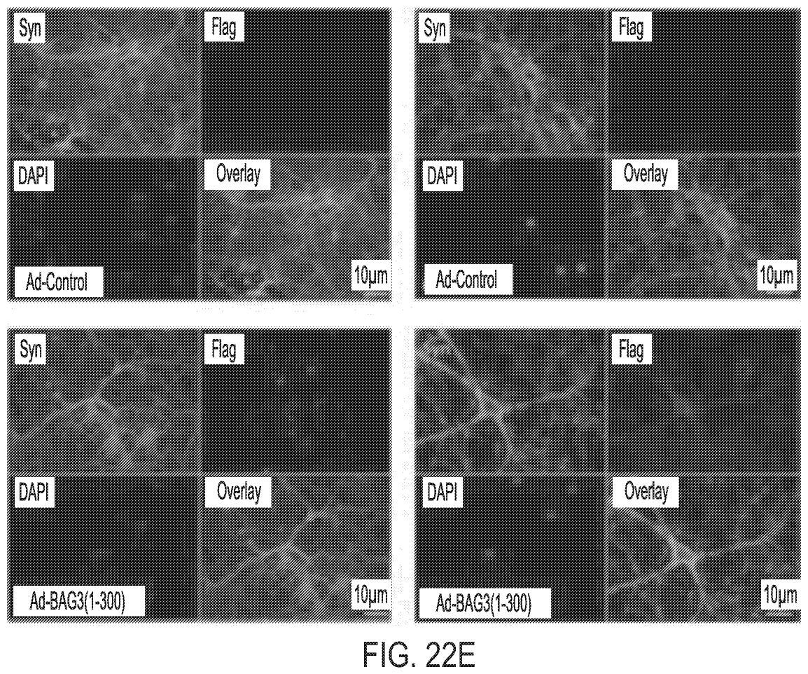

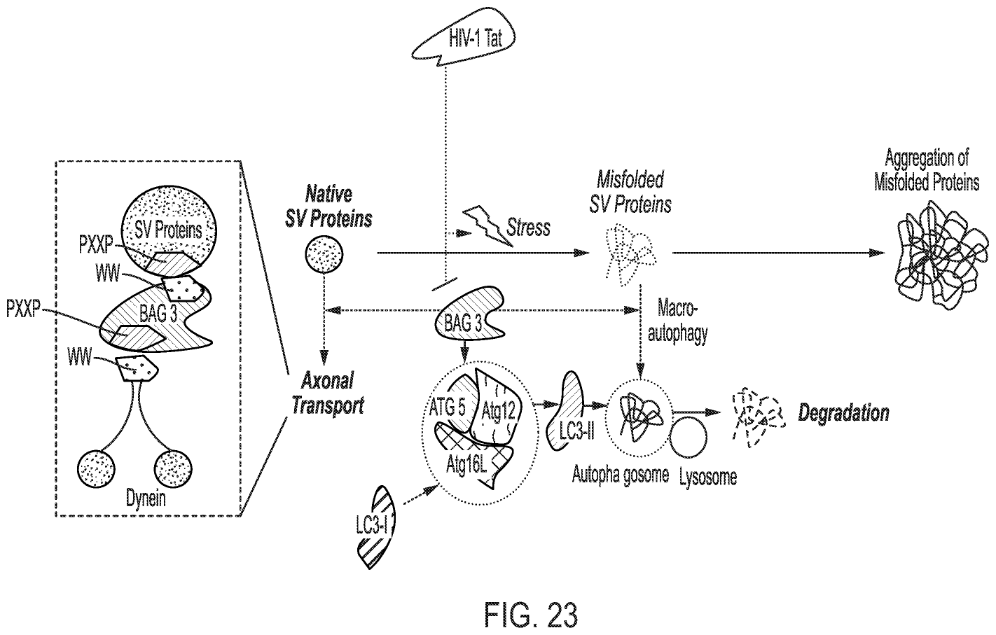

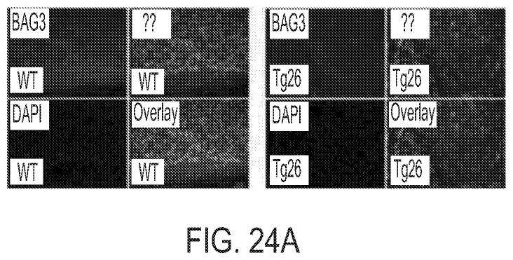

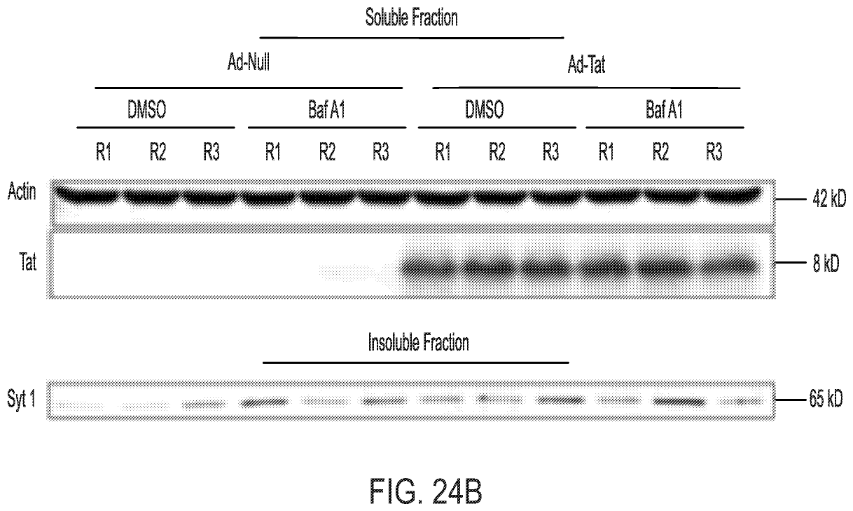

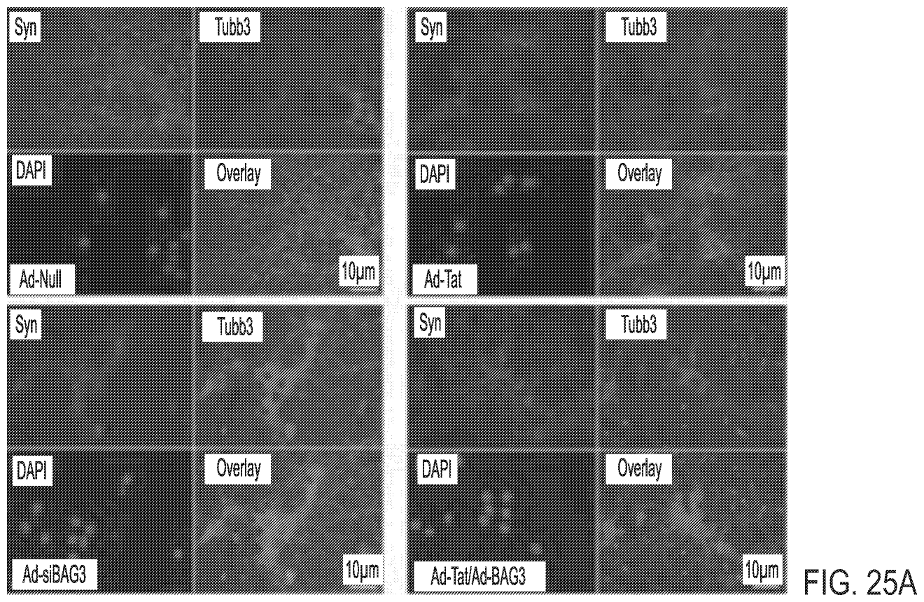

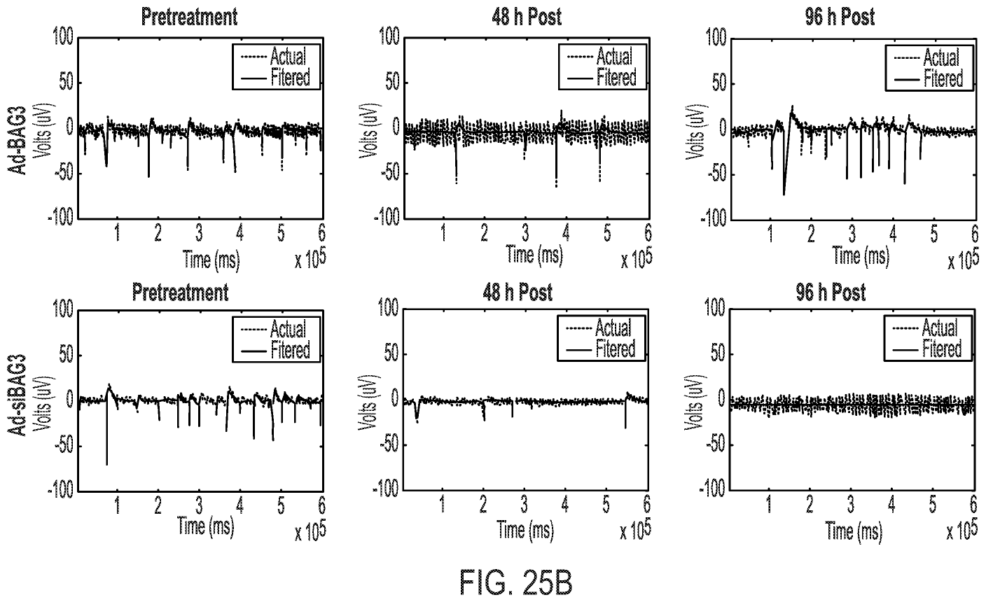

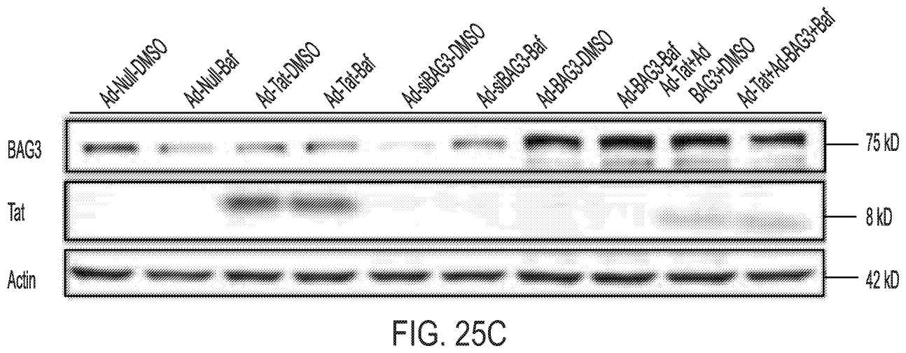

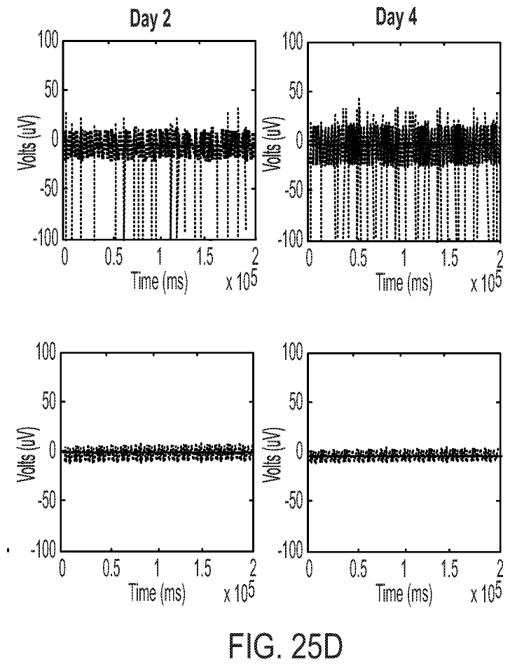

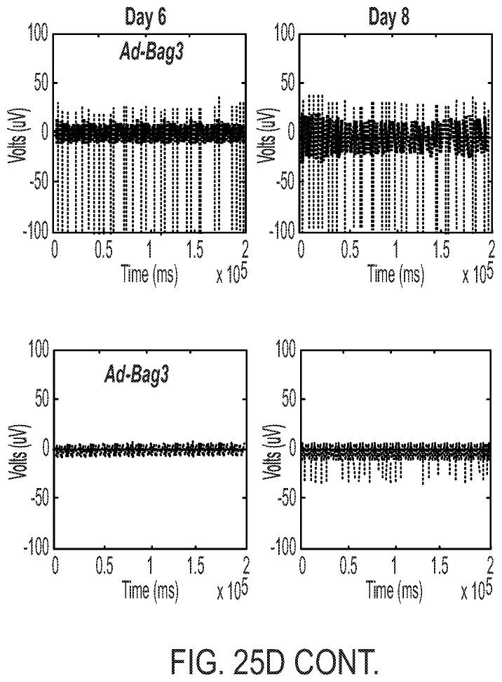

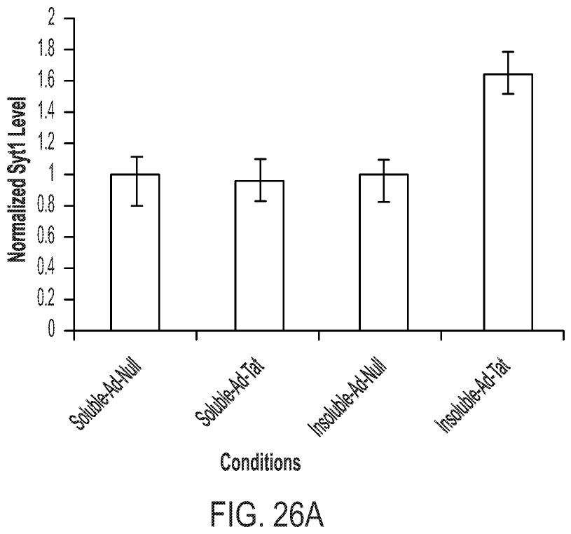

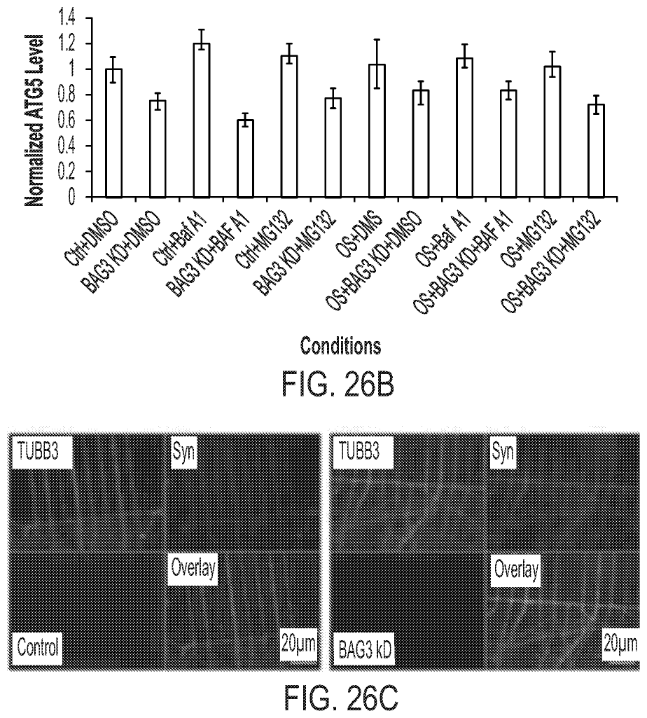

Results from immunocytochemistry experiments showed that in the absence of Tat, BAG3 and Tom20 are found in the cytoplasm and co-localize in the perinuclear region. In the presence of Tat, large numbers of puncta containing both BAG3 and Tom20 are seen throughout the cytoplasm, showing changes in localization of BAG3 and Tom20 in the cells under stress induced by the presence of Tat (FIGS. 7A, 7B).

Results from experiments showed that Tat effects subcellular co-location of BAG3 with Parkin in hippocampal neurons (FIGS. 8A, 8B). Immunocytochemistry shows BAG3 localization to both the cytoplasmic and nuclear compartments. Co-localization of Parkin and BAG3 in the cytoplasm of neuronal cells was seen, more specifically localizing to puncta in the perinuclear region FIG. 8A). This colocalization was disrupted upon treatment with 50 nM of recombinant Tat (rTat).

HIV-1 Tat also promotes the initiation of mitophagy signaling by increasing levels of mitophagy-associated proteins (FIG. 11). Primary neurons were treated with Tat (50 nM) and subfractioned by differential centrifugation to assess sub-cellular localization. As seen, in the presence of Tat, Parkin (PARK2) immunoreactivity was increased in the mitochondrial fraction. Examination of PINK1 levels by Western blot showed a modest increase in the level of proteins corresponding to two isoforms of PINK1 CoxIV was used as a mitochondrial loading control and tubulin was used as a cytosolic loading.) HIV-Tat was also shown to reduce ATP generation (FIG. 12). To evaluate energy production and availability in neurons, where functional mitochondria are critical, the level of ATP were determined in primary neurons following treatment with Tat. An ATP-luciferase assay revealed a substantial decrease in the level of ATP production in cells following Tat treatment. Heat inactivation (hi) and rotenone were as controls.

HIV-Tat was shown to increase mtDNA lesions and inhibits SOD activity (FIG. 13). Treatment of primary neuronal cells with HIV-1 Tat induced mitochondrial DNA (mtDNA) damage as detected by a decrease in the intensity of the amplified 10 k DNA fragment from mtDNA, which correlates with a decrease in the activity of superoxide dismutase (SOD), indicative of reduced antioxidant capacity and increased susceptibility to superoxide-mediated stress.

### Example 2: Perturbation of Synapsins Homeostasis Through HIV-1 Tat-Mediated Suppression of BAG3 in Primary Neuronal Cells

HIV-1 does not infect neurons but promotes neurodegenerative processes through release of HIV-1 neurotoxic proteins, such as Nef and Tat, from infected bystander cells that subsequently undergo endocytosis by neurons (2). Tat is a known neurotoxic agent, as it alters bioenergetics and survival pathways of neurons like cholesterol homeostasis (3) and leads to alterations in dopamine secretion (4) and synaptic loss (5). Furthermore, Tat has been shown to reduce neuronal excitability, damage dendritic spines (6) and potentiate inhibitory GABAA receptors (7). It also affects synaptodendritic stability (8) and induces cell death and synapse loss through NMDA (N-methyl-D-aspartate) receptor activation (9).

In the work herein, the effects of HIV-1 Tat on lysosomal autophagy was studied, a major stress-induced PQC pathway in neurons, and the role of a key molecular regulator of chaperone activity, BAG3, on proteostasis in the context of turnover of markers of synaptic vesicles (SVs) such as synapsins and syanptotagmin 1 (Syt1). The biochemical assays described herein, clearly indicated the detrimental effect of HIV-1 Tat on the normal distribution of synapsis essential to neuronal signal transmission. Central to this phenomenon, it was discovered that levels of BAG3 mRNA and protein were significantly suppressed in the presence of Tat, further indicating downregulation of ATG5 protein and LC3-4 lipidation to LC3-II, crucial steps in the cellular autophagy response to oxidative stress induced by Tat. This cascade of events led to the accumulation of synapsin positive puncta in the neuronal soma and processes proximal to it. Besides this autophagy-derived phenomenon, the interaction of BAG3 with synaptic vesicle proteins was confirmed, which probably serves a dual function: to transport SV proteins along axons and to sequester the aberrant synapsins and Syt1 through macroautophagy.

Experimental Procedures

Tissue and Cell Culture

Tissue preparation and cell cultures were performed according to the guidelines approved by Temple University Institutional Animal Care and Use Committee. Tat transgenic mice Doxycycline (DOX)-inducible GFAP promoter driven HIV-1 Tat transgenic mice were provided by the Comprehensive NeuroAIDS Center. Adult mice (18-28 g) were singly housed in a temperature (21-23° C.) and humidity-controlled vivarium with constant airflow on a reverse 12-h light/dark cycle (lights off at 09:00). Food and water were available ad libitum. Mice expressed the HIV-1 tat transgene (TAT) or their control littermates. Briefly, TAT mice conditionally-expressed the HIV-1 Tati-se protein in an astrocyte-specific manner under the control of a GFAP-driven Tet-on promoter which is activated in the presence of DOX. TAT mice received an 80 mg/kg intraperitoneal (i.p.) injection of doxycycline hyclate (DOX). After this 14-day period, all mice were euthanized and prepared for harvest of brain tissue for western analyses.

Tg26 Mice

Tg26 HIV transgenic mice are a well described mouse model that encode the entire pNL4-3 HIV-1 genome minus a segment of gag/pol genes. This homozygous mouse expresses HIV viral proteins such as Tat and Nef and exhibits neuropathology similar to HIV-associated symptoms such as neurocognitive disorders and cardiac problems (59).

Primary Rat Neuronal Culture

Dissociated cell cultures of hippocampal and cortical neurons were prepared using dissected E18 prenatal rat embryonic brains. After digestion in 0.25% trypsin solution, neurons were plated on tissue culture plates and slides pre-coated with poly-D-lysine (Sigma, St. Louis, Mo.) and laminin (Invitrogen 23017, USA). Cells were maintained 14 days prior to treatments. Neurons were transduced with Ad-Null (Vector Biolabs, Malvern, Pa.), Ad-Tat (made in-house), Ad-siBAG3 (Vector Biolabs, Malvern, Pa.) and Ad-BAG3 (Vector Biolabs, Malvern, Pa.) with MOI 1 to 4 or recombinant Tat protein (50 ng/ml, full length, 101 amino acids, Immunodiagnostics, MA). Tat bioactivity was verified by the LTR-luciferase assay. MG132 (5 μM) and Bafilomycin A 1 (50 nM, Sigma) were applied for 4 h prior to lysis to block proteasome and lysosomal autophagy, respectively.

Immunoblotting

Neuronal and mouse brain tissue lysates were prepared using RIPA lysis buffer (whole cell extract), Tris-Triton (soluble fraction) or 2% SDS in PBS (insoluble fraction), all containing protease inhibitor cocktail (Sigma-Aldrich, St. Louis, Mo.). Protein concentration was measured using Bio-Rad protein assay reagent (Bio-Rad, Hercules, Calif.). SDS-polyacrylamide gels, 10-12%, and nitrocellulose membranes (LI-COR, Inc., Lincoln, Nebr.) were used for electrophoretic protein separation and transfer, respectively. Membranes were blocked (overnight at 4° C.) in Odyssey (LI-COR) blocking buffer and incubated with primary and secondary (I h at RT) antibodies. Membranes were scanned using an Odyssey1 CLx Imaging System (LI-COR, Inc.) The following primary antibodies were used for Western blotting: BAG3 (Proteintech, Rosemont, Ill., 10599-1-AP), Synapsin (Cell Signaling, D12G5), Synaptotagmin 1 (Santa Cruz, sc-136480), Tat (NIH AIDS Reagent Program, Germantown, Md., R705), LC3 (Sigma, L8918), ATG5 (Abcam, 108327), β3-Tubulin (Sigma, T8578), Bcl-xl (Santa Cruz, sc-8392), β-Actin (Santa Cruz, sc-47778), VCP (Santa Cruz, sc-20799), Beclin-1 (Cell Signaling Technology, 3738), Parkin (Abcam, ab77924), Tomm20 (Abcam, ab199641).

Immunocytochemistry

Intracellular Tat was probed in Ad-Tat transduced neurons through immunocytochemistry. Neurons were transduced with Ad-Tat and Ad-Null on 14 DIV with MOI 1. After 72 hr, the cells were fixed and permeabilized using −20° C. cooled acetone (Sigma). Following blocking with (1%) BSA in PBST, the neurons were labeled with the following antibodies (1:100): BAG3 (Proteintech, Rosemont, Ill., 10599-1-AP), Synapsin (Cell Signaling, D12G5), Synaptotagmin 1 (Santa Cruz, sc-136480), β3-Tubulin (Sigma, T8578), ATG5 (Abeam, 108327), Hsc70 (Enzo, N27F3-4), Ubiquitin (Santa Cruz, sc-8017) and rabbit polyclonal Tat antibodies (NIH AIDS Reagent Program, Germantown, Md., R705). ALEXA FLUOR® secondary antibodies (ThermoFisher Scientific, OR) and VECTASHIELD medium (Vector Laboratories, Burlingame, Calif.) were used for labeling and mounting, respectively. Images were prepared via Leica fluorescent microscope (Leica Microsystems, IL).

Immunohistochemistry

Brain tissue samples of wild type and Tg26 transgenic mice were collected and frozen tissue sectioning was embedded based on standard protocol. The thickness of sections was 9 μm. Sections were probed with anti-BAG3 (1:250) and anti-β3-tubulin (1:500) antibodies. The fluorescent staining followed the same procedure as described for immunocytochemistry.

Cell Culture on Microfluidic Device

Rat E18 hippocampal neurons were plated on microfluidic devices (Xona Microfluidics, TCND500) placed on glass cover slips (Leica Biosystems) coated with poly-D-lysine and laminin as described above. Neurons 14 DIV were transduced with Ad-Null and Ad-siBAG3. After 72 h cells were fixed and permeabilized in −20° C. cooled acetone (Sigma). The rest of the steps were similar to immunocytochemistry for labeling and imaging with synapsins and β3-tubulin.

RNA Isolation and cDNA Preparation

Following treatment, total RNA for each condition was extracted using the Trizol (ThermoFisher, Carlsbad, Calif.) extraction protocol followed by RNA cleaning protocol using DIRECT-ZOL™ RNA MiniPrep Plus (Zymo Research, CA, USA) following the manufacturer's recommendations. cDNA synthesis on total RNA samples was performed with High Capacity cDNA Reverse Transcription Kit (Thermo Fisher Scientific) according to manufacturer's protocol.

Real-Time Quantitative RT-PCR (qRT-PCR)

Specific primers for the BAG3 transcriptome were designed considering the exon junction positions in the genomic DNA (BAG3 Forward primer: 5′-GGCCCTAAGGAAACTGCAT-3′ SEQ ID NO: 1; Reverse primer: 5′-GGGAATGGGAATGTAACCTG-3′; SEQ ID NO: 2). The specificity and efficiency of the primers were checked by RT-PCR using Q5 High-Fidelity PCR Kit (New England Biolabs). All qPCR reactions were performed with the LIGHTCYCLER96® (Roche) using the SYBR™ Green master mix (Applied Biosystems, ThermoFisher), according to manufacturer's protocol. Relative quantity was normalized to Actin expression.

Microelectrode Array-Based Electrophysiology

Microelectrode arrays (MEAs) were used to perform electrophysiological recordings at 2 kHz on an MEA60 system (Multichannel systems, Germany) from hippocampal neurons undergoing control, BAG3 KD, BAG3 overexpression, and Tat expression starting from 25 DIV.

Results

Neuronal synaptic network is disrupted in the presence of HIV-1 Tat: Transduction of HIV-1 Tat protein via an adenoviral vector significantly alters cellular distribution of synapsins. The immunocytochemical labeling of synaptic vesicles in rat hippocampal neurons shows that Tat expression leads to accumulation of synapsins positive puncta close to the soma rather than more widespread distribution throughout the neuronal axon terminals (FIGS. 18A, 18B). Further investigation of the synaptic vesicle proteins, synapsins and Syt1 using Synaptotagmin 1, another neuronal synaptic vesicle marker, is similarly located close to the soma upon expression of Tat (FIG. 18C). Further investigation of the synaptic vesicle proteins, synapsins and Syt1 using Neuronal synaptic network is disrupted in the presence of HIV-1 Tat transduction of HIV-1 Tat protein via an adenoviral vector significantly alters cellular distribution of synapsins. The immunocytochemical labeling of synaptic vesicles in rat hippocampal neurons shows that Tat expression leads to accumulation of synapsins positive puncta close to the soma rather than more widespread distribution throughout the neuronal axon terminals (FIGS. 18A, 18B). Synaptotagmin 1, another neuronal synaptic vesicle marker, is similarly located close to the soma upon expression of Tat (FIG. 18C). Further investigation of the synaptic vesicle proteins, synapsins and Syt1 using immunoblotting indicated that Tat not only alters synaptic distribution, but the proteins present in different cellular compartments (FIGS. 18D, 18E). After fractionation of soluble (cytosolic) and insoluble (membrane, nuclei-related and aggregates) protein extracts, r synapsins and Syt1 were probed for (FIGS. 18D, 18E). In both cases, a significant increase (over 70%) in protein levels was observed in the insoluble fraction upon Tat expression, providing evidence that Tat promotes formation of synaptic protein aggregates. To assess the dose dependent impact of Tat on synapsins-positive aggregates and subcellular localization, hippocampal neurons were exposed to different concentrations of recombinant Tat (rTat) protein. Results indicated that with increasing Tat concentration, increased synapsin-positive aggregates proximal to the neuronal soma were observed (FIG. 18F). This aggregation appears in the form of puncta in the cell body and processes proximal to soma (FIGS. 18C, 18F).

Tat downregulates BAG3 protein levels by decreasing its mRNA levels: Tat expression significantly suppressed BAG3 protein levels in both hippocampal and cortical neurons (FIG. 2A). Time course studies of Tat expression revealed that Tat gradually diminishes BAG3 in the whole-cell extract in neurons. This phenomenon was observed starting 48 h post-transduction and continued until complete disappearance of BAG3 around day 6 post-transduction (FIGS. 19B, 19C). Similarly, DOX-induced Tat expression in the brains of Tat-transgenic mice, led to a decrease in BAG3 in the whole brain protein lysate (FIG. 19D). Tat expression also blocked the adenoviral overexpression of BAG3 (FIG. 19E). To determine the mechanism underlying the Tat-mediated reduction of BAG3, a series of experiments were conducted to assess rates of transcription and translation. It was first hypothesized that Tat might cause BAG3 degradation through different proteolysis pathways. To that end, the two major protein degradation pathways, proteosomal and lysosomal pathways were separately blocked using appropriate inhibitors. MG132 was used to inhibit the proteasome pathway for 4 h on day 3 post-transduction. Since BAG3 levels were not altered upon MG132 application and thus proteasome inhibition, the role of this degradation pathway was ruled out (FIG. 19F). A second major protein degradation pathway was examined by application of Bafilomycin A1 (Baf A1) to inhibit lysosomal degradation. Once again, no significant changes in BAG3 levels were observed, providing evidence that lysosomal degradation was unlikely to be involved in Tat-mediated BAG3 decline further implicating transcriptional or post transcriptional regulation (FIG. 19G). To examine this hypothesis, BAG3 mRNA levels were assessed in the presence and absence of Tat expression in hippocampal neurons. The results demonstrated that Tat downregulated BAG3 mRNA (FIG. 19H). To examine the stability of BAG3 mRNA, transcription was blocked with Actinomycin D and BAG3 mRNA levels were quantified in control and Tat expressing neurons (FIG. 19I). The results provided evidence that the BAG3 mRNA half-life, despite the multiple fold lower basal level under Tat expression, did not change significantly. Finally, neurons were treated with various concentrations of rTat protein (50, 100, and 200 ng/ml) and observed the same inhibitory effect on BAG3 mRNA (FIG. 19J).

BAG3 suppression promotes formation of aggregates containing synaptic vesicle proteins: As observed with Tat-mediated localization of synapsin-positive puncta to the neuronal soma, siRNA-mediated BAG3 knockdown had a similar effect on synapsin distribution by promoting its accumulation in the soma proximity (FIGS. 20A, 20B). Further, Western blot analysis following BAG3 knockdown (KD) demonstrated an aggregation of Syn and Syt1 in the insoluble fraction (FIGS. 20C, 20D). Accumulation of Syt 1 appeared to be affected more by the BAG3 KD compared to the autophagy inhibition. This phenomenon could be because of the longer half-life of the protein compared to the duration of the Baf A1 treatment. The aggregation of synapsin-positive puncta under BAG3 knockdown was further investigated using a microfluidic device generating micrometer width grooves. Neurons were plated on a built-in set of wells and processes extended along the grooves to allow for assessment of axonal aggregation of synapsins. Knockdown of BAG3 led to the formation of large aggregates within the axons (FIGS. 20E, 20F and 26C), as compared to control. Quantification of the aggregated synapsins revealed that BAG3 KD resulted in the formation of 1.9-fold as many synapsin-positive aggregates compared to the control (FIG. 20E, bottom row). Finally, adenoviral-mediated expression of HIV-1 Tat in cortical neurons resulted in the accumulation of aggregated Syt1 in the insoluble proteins fraction under control and lysosomal autophagy inhibition (FIG. 24B).

HIV-1 Tat and BAG3 suppression downregulate ATG5 and inhibit lysosomal autophagy: To investigate the possible mechanisms underlying SV protein aggregate formation under BAG3 KD and Tat expression, the lysosomal protein degradation pathway was studied under BAG3 KD and oxidative stress to simulate the effects of Tat. BAG3 KD led to a significant reduction in LC3-II formation and autophagy flux under the control and H₂O₂-induced oxidative stress conditions (FIG. 21A). Co-immunoprecipitation evidenced an association between BAG3 and LC3 (FIG. 21B) and BAG3 and ATG5 (FIG. 21D), an essential protein for LC3-I lipidation and conversion to LC3-II is required for autophagosome formation. The data indicated that BAG3 KD, under proteasome and lysosome inhibition and during oxidative stress, led to ATG5 downregulation (FIGS. 21C and 26A-26C). BAG3 KD adversely affected ubiquitinated protein levels under oxidative stress; while the presence of oxidative stress shift the ubiquitination flux from the proteasome to lysosome under the control neurons, BAG3 KD impairs this shift (FIG. 21E). ATG5 co-localized with Hsc70, a canonical heat shock protein and a BAG3 partner, as determined by immunocytochemistry imaging (FIG. 21F). BAG3 was also shown to co-localize with ubiquitin and/or ubiquitinated proteins (FIG. 21G). Finally, Tat expression downregulated ATG5 levels in a time-dependent manner (FIG. 21H), with no effect on its mRNA levels (FIG. 21I).

BAG3 interacts with synaptic vesicle proteins: It was next examined whether BAG3 interacts with synaptic vesicle proteins including synapsins and Syt1 by utilizing different biochemical assays. Co-immunoprecipitation with synapsins antibody and probing for BAG3 clearly indicated BAG3 interaction with synapsins in both hippocampal and cortical neurons, under both normal and H₂O₂-induced oxidative stress (FIG. 22A). co-immunoprecipitation with BAG3 antibody resulted in pulldown of synaptotagmin 1 (FIG. 22B). As depicted in FIG. 5c, BAG3 co-localizes with Syt1 in rat hippocampal neurons. To examine whether there is an interaction between BAG3 WW domain and proline rich domains of synapsins and if this would affect synaptic vesicle proteins distributions, a BAG3 mutant containing only the WW domain and the IPV motif (BAG3 1-300) was overexpressed using an adenovirus vector. As shown in FIGS. 22D, 22E, overexpression of the WW domain without the PXXP domain of BAG3, which mediates its interaction with motor protein dynein, disrupts the normal synapsins distribution and leads to a similar effect as observed in BAG3 KD and Tat expression.

### DISCUSSION

In this study, it was shown that HIV-1 Tat-induced alterations in synaptic vesicle proteins, namely synapsins and synaptotagmin 1. These effects influenced not only the distribution of these proteins in neurons but also the total levels of synapsin protein family members in the soluble and insoluble protein fractions of neuronal cell lysate. Recently, it has been shown that synaptic vesicles were transported along axons via fast and slow axonal transport mechanisms and could involve Hsc70 (a member of Hsp70 family) chaperone activity (52, 53). BAG3 is a regulator of Hsp70 chaperone activity through utilizing motor protein dynein to direct degradable substrates to aggresomes in an ubiquitin-independent manner (54). Conversely, inhibition of dynein-dependent retrograde transport of aggregated proteins to perinucleus aggresomes diverted neurons to activate proteasomal autophagy and reduced protein aggregates in a BAG1 dependent manner with implications in proteinopathy in motor neuron diseases (55).

A comprehensive set of experiments were performed to study the effects of HIV-1 Tat on BAG3. The data clearly demonstrate the negative impact of Tat on BAG3 homeostasis in neuronal cells, both in dissociated rat cortical and hippocampal neurons and in Tat-transgenic mice and Tg26 mice brain slices. The inhibitory effect of Tat on BAG3 was not restored by interfering with lysosomal and proteasomal autophagy pathways, providing evidence that Tat did not affect BAG3 through protein destabilization. The qRT-PCR data showed that Tat expression using an adenoviral vector as well as treatment of neurons with recombinant Tat reduced the BAG3 mRNA level. Tat application together with overexpression of BAG3 using a different promoter (CMV) also led to reduced levels of BAG3 protein. These observations provide evidence that Tat suppresses BAG3 through a post-transcriptional effect on BAG3 mRNA. To examine the potential role of BAG3 in the homeostasis of synapsins, BAG3 was knocked down using a BAG3 siRNA. Similar to what was observed following Tat treatment, BAG3 KD led to the accumulation of synapsins in the proximity neuronal soma and aggregation of synapsins along axons grown in the microgroove device. Both synapsins and another synaptic vesicle marker, synaptotagmin 1, were shown to accumulate in the insoluble fraction of cell lysate following BAG3 KD. This phenomenon is hypothesized to partially occur through impairment of degradation of obsolete synaptic proteins and/or impairment of synaptic vesicle proteins axonal transport mediated by direct interaction with BAG3. Through its multi-domain structure, BAG3 is engaged in diverse proteome homeostasis processes. These complexes can target proteins for degradation, ubiquitination and further transport them along the microtubule towards aggresome formation. SQSTM1 helps BAG3-associated protein aggregates be engulfed by phagophore through binding to LC338. BAG3 overexpression has been observed along with heat and mechanical stress that activate chaperone assisted autophagy mechanisms (56). This process can be induced by inhibiting proteasome, which consequently leads to the activation of BAG3-dependent lysosomal autophagy and LC3-4 to LC3-II conversion (29). BAG3 depletion results in reduced levels of LC3-II and lysosomal autophagy47. LC3-II is reduced upon BAG3 KD; when ATG5 is knocked-down, BAG3 overexpression cannot restore LC3-II (38) levels. In accordance with these earlier observations, an interaction between BAG3 with both LC3 and ATG5 is shown herein. Furthermore, it was demonstrated herein that BAG3 KD led to decreased levels of ATG5 and LC3-II formation under normal conditions and oxidative stress. The results indicated co-localization of BAG3 with ubiquitinated aggregates, possibly aggresomes, in the neuronal soma. More interestingly, following BAG3 KD, it appeared that neuronal ubiquitination machinery did not respond to oxidative stress. Furthermore, Tat expression reduced the level of ATG5 in a time dependent manner, similar to BAG3 suppression (FIGS. 21A-21I). Collectively, it can be concluded that BAG3 is essential for autophagy of ubiquitinated proteins in general and the LC3-II lipidation and lysosomal autophagy through maintaining the levels of ATG5 protein, in particular (ATG5 mRNA was not affected by BAG3 KD or Tat expression). To examine the interaction of BAG3 and synaptic vesicle proteins, co-immunoprecipitation assays were performed using BAG3 and Syn antibodies and probed with Syt1 and BAG3 antibodies, respectively. In both cases, the interaction of BAG3 and synaptic vesicle proteins was confirmed (FIGS. 22A-22E). BAG3 also co-localized with synaptotagmin 1 in immunocytochemistry assay. BAG3 PXXP domain helps it bind to dynein, giving it vesicle transport activity of SH3 domain containing proteins including signal transducing adapter proteins and a broad range of protein kinases. WW domain of BAG3 mediates interactions with PXXP domain of other proteins (57) and possibly BAG3-BAG3 communication (58). BAG3, in turn, utilizes its WW domain to interact with the proline rich domain (e.g., PPPY and PPSY) of the target proteins (56). As all synapsin proteins possess a proline-rich domain, it was examined whether this domain interacted with the WW domain of BAG3, facilitating its transport via dynein retrograde axonal transport mechanism. To this end, a BAG3 (1-300) mutant containing only the WW domain (with no PXXP to interact with dynein) was overexpressed to study the effect on synaptic vesicles proteins as probed by synapsins. This overexpression resulted in comparable observations as in BAG3 KD and Tat expression. Moreover, it was also shown herein that BAG3 overexpression restores the effect of Tat on synaptic vesicle distribution as well as neuronal electrophysiological activity measured by microelectrode arrays upon BAG3 KD and Tat expression. In conclusion, a mechanism depicted in FIG. 23 is proposed, where BAG3 interacts with synapsins and is essential for the homeostasis of these proteins as well as healthy state of synaptic vesicle proteins. The BAG3 suppression imposed by HIV-1 Tat in neurons has detrimental effects on SV proteins distribution and possibly synapse formation. These observations have implications in synaptic loss and alterations in neuronal plasticity as a result of HIV-1 infection and Tat expression.

### REFERENCES

1. Dahal, S., Chitti, S. V., Nair, M. P. & Saxena, S. K. Interactive effects of cocaine on HIV infection: implication in HIV-associated neurocognitive disorder and neuro-AIDS.

Front. Microbiol. 6, 931 (2015).
2. King, J., Eugenin, E., Buckner, C. & Berman, J. HIV tat and neurotoxicity. Microb. Infect. 8, 1347-1357 (2006).
3. Ahooyi, T. M. et al. Dysregulation of neuronal cholesterol homeostasis upon exposure to HIV-1 Tat and cocaine revealed by RNA-sequencing. Sci. Rep. 8, 16300 (2018).
4. Yuan, Y. et al. Molecular mechanism of HIV-1 Tat interacting with humandopamine transporter. ACS Chem. Neurosci. 6, 658-665 (2015).
5. Haughey, N., Nath, A., Mattson, M., Slevin, J. & Geiger, J. HIV-1 Tat throughphosphorylation of NMDA receptors potentiates glutamate excitotoxicity. J. Neurochem. 78, 457-467 (2001).
6. Fitting, S. et al. Synaptic dysfunction in the hippocampus accompanies learning and memory deficits in human immunodeficiency virus type-1 Tat transgenic mice. Biol.

Psychiatry 73, 443-453 (2013).
7. Mohseni Ahooyi, T. et al. Network analysis of hippocampal neurons by microelectrode array in the presence of HIV-1 Tat and cocaine. J. Cell Physiol. 233, 9299-9311 (2018).
8. Bertrand, S. J., Aksenova, M. V., Mactutus, C. F. & Booze, R. M. HIV-1 Tat protein variants: critical role for the cysteine region in synaptodendritic injury. Exp. Neurol. 248, 228-235 (2013).
9. Krogh, K. A. The Mechanism of HIV-1 Tat-induced Changes in NMDA Receptor Function. (University of Minnesota, Minnesota, 2014).
10. Ho, L. et al. Altered expression of a-type but not b-type synapsin isoform in the brain of patients at high risk for Alzheimer's disease assessed by DNA microarray technique. Neurosci. Lett. 298, 191-194 (2001).
11. Hartl, D. et al. SORLA regulates calpain-dependent degradation of synapsin. Alzheimers Dement. 12, 952-963 (2016).
12. Liu, A. et al. Regulation of neurotransmitter release by amyloid precursor protein through synapsin phosphorylation. Neurochem. Res. 44, 683-691 (2019).
13. Qin, S., Hu, X.-Y., Xu, H. & Zhou, J. N. Regional alteration of synapsin I in the hippocampal formation of Alzheimer's disease patients. Acta Neuropathol. 107, 209-215 (2004).
14. Zaltieri, M. et al. alpha-synuclein and synapsin III cooperatively regulate synaptic function in dopamine neurons. J. Cell Sci. 157867 (2015).
15. Faustini, G. et al. Synapsin III deficiency hampers α-synuclein aggregation, striatal synaptic damage and nigral cell loss in an AAV-based mouse model of Parkinson's disease. Acta Neuropathol. 136, 621-639 (2018).
16. Longhena, F. et al. Synapsin III is a key component of α-synuclein fibrils in Lewy bodies of PD brains. Brain Pathol. 28, 875-888 (2018).
17. Liévens, J.-C., Woodman, B., Mahal, A., Bates, G. P. J. M. & Neuroscience, C. Abnormal phosphorylation of synapsin I predicts a neuronal transmission impairment in the R6/2 Huntington's disease transgenic mice. Mol. Cell Neurosci. 20, 638-648 (2002).
18. Porton, B. & Wetsel, W. C. Reduction of synapsin III in the prefrontal cortex of individuals with schizophrenia. Schizophr. Res. 94, 366-370 (2007).
19. Saviouk, V., Moreau, M. P., Tereshchenko, I. V. & Brzustowicz, L. M. Association of synapsin 2 with schizophrenia in families of Northern European ancestry. Schizophr. Res. 96, 100-111 (2007).
20. Dyck, B. A., Beyaert, M. G., Ferro, M. A. & Mishra, R. K. Medial prefrontal cortical synapsin II knock-down induces behavioral abnormalities in the rat: exa synapsin II in the pathophysiology of schizophrenia. Schizophr. Res. 130, 250-259 (2011).
21. Molinaro, L., Hui, P., Tan, M. & Mishra, R. K. Role of presynaptic phosphoprotein synapsin II in schizophrenia. World J. Psychiatry 5, 260 (2015).
22. Greco, B. et al. Autism-related behavioral abnormalities in synapsin knockout mice. Behav. Brain Res. 251, 65-74 (2013).
23. Provenzano, G. et al. Reduced phosphorylation of synapsin I in the hippocampus of Engrailed-2 knockout mice, a model for autism spectrum disorders. Neuroscience 286, 122-130 (2015).
24. Michetti, C. et al. The knockout of Synapsin II in mice impairs social behavior and functional connectivity generating an ASD-like phenotype. Cereb. Cortex. 27, 5014-5023 (2017).
25. Tang, L. T.-H., Craig, T. J. & Henley, J. M. SUMOylation of synapsin 1a maintains synaptic vesicle availability and is reduced in an autism mutation. Nat. Commun. 6, 7728 (2015).
26. Thonsranoi, K. et al. Increased synapsin I expression in cerebral malaria. Int. J. Clin. Exp. Pathol. 8, 13996 (2015).
27. Das, C. K. et al. BAG3 overexpression and cytoprotective autophagy mediate apoptosis resistance in chemoresistant breast cancer cells. Neoplasia 20, 263-279 (2018).
28. Merabova, N. et al. WW domain of BAG3 is required for the induction of autophagy in glioma cells. J. Cell Physiol. 230, 831-841 (2015).
29. Liu, B.-Q. et al. BAG3-dependent noncanonical autophagy induced by proteasome inhibition in HepG2 cells. Autophagy 9, 905-916 (2013).
30. Gamerdinger, M. et al. Protein quality control during aging involves recruitment of the macroautophagy pathway by BAG3. EMBO J. 28, 889-901 (2009).
31. Tahrir, F. G. et al. Evidence for the role of BAG3 in mitochondrial quality control in cardiomyocytes. J. Cell Physiol. 232, 797-805 (2017).
32. Kim, Y. H., Kim, K. Y., Kim, J.-S. & Kim, Y. H. Inhibition of autophagy enhances dynamin inhibitor-induced apoptosis via promoting Bak activation and mitochondrial damage in human Jurkat T cells. Biochem. Biophys. Res. Commun. 478, 1609-1616(2016).
33. Lee, J.-H. et al. Bis, a Bcl-2-binding protein that synergizes with Bcl-2 in preventing cell death. Oncogene 18, 6183 (1999).
34. Inomata, Y. et al. Bcl-2-associated athanogene 3 (BAG3) is an enhancer of small heat shock protein turnover via activation of autophagy in the heart. Biochem. Biophys. Res. Commun. 496, 1141-1147 (2018).
35. Ulbricht, A. et al. Cellular mechanotransduction relies on tension-induced and chaperone-assisted autophagy. Curr. Biol. 23, 430-435 (2013).
36. Kyratsous, C. A. & Silverstein, S. J. BAG3, a host cochaperone, facilitates varicellazoster virus replication. J Virol. 81, 7491-7503 (2007).
37. Rosati, A. et al. Evidence for BAG3 modulation of HIV-1 gene transcription. J.

Cell. Physiol. 210, 676-683 (2007).
38. Cao, Y.-L. et al. A role of BAG3 in regulating SNCA/α-synuclein clearance via selective macroautophagy. Neurobiol. Aging 60, 104-115 (2017).
39. Klimek, C., Kathage, B., Wördehoff, J. & Höhfeld, J. BAG3-mediated proteostasis at a glance. J. Cell Sci. 130, 2781-2788 (2017).
40. Paola Bruno, A. et al. BAG3 mRNA is present in synaptosomal polysomes of rat brain. Cell Cycle 13, 1357-1357 (2014).
41. Gentilella, A. & Khalili, K. BAG3 expression is sustained by FGF2 in neural progenitor cells and impacts cell proliferation. Cell Cycle 9, 4245-4247 (2010).
42. Rusmini, P. et al. The role of the heat shock protein B8 (HSPB8) in motor neuron diseases. Front. Mol. Neurosci. 10, 176 (2017).
43. Stirner, E. & Behl, C. The role of the multifunctional BAG3 protein in cellular protein quality control and in disease. Front. Mol. Neurosci. 10, 177 (2017).
44. Franceschelli, S. et al. Bag3 gene expression is regulated by heat shock factor 1. J. Cell Physiol. 215, 575-577 (2008).
45. Echaniz-Laguna, A. et al. Axonal neuropathies due to mutations in small heat shock proteins: clinical, genetic, and functional insights into novel mutations. Hum. Mutat. 38, 556-568 (2017).
46. Bruno, A. P. et al. Identification of a synaptosome-associated form of BAG3 protein. Cell Cycle 7, 3104-3105 (2008).
47. Carra, S., Seguin, S. J., Lambert, H. & Landry, J. HspB8 chaperone activity toward poly (Q)-containing proteins depends on its association with Bag3, a stimulator of macroautophagy. J. Biol. Chem. 283, 1437-1444 (2008).
48. Tang, M., Ji, C., Pallo, S., Rahman, I. & Johnson, G. V. Nrf2 mediates the expression of BAG3 and autophagy cargo adaptor proteins and tau clearance in an age-dependent manner. Neurobiol. Aging 63, 128-139 (2018).
49. Fontanella, B. et al. The co-chaperone BAG3 interacts with the cytosolic chaperonin CCT: new hints for actin folding. Int. J. Biochem. Cell Biol. 42, 641-650 (2010).
50. Renziehausen, J. et al. The cleavage product of amyloid-β protein precursor sAβPPα modulates BAG3-dependent aggresome formation and enhances cellular proteasomal activity. J. Alzheimers Dis. 44, 879-896 (2015).
51. Santoro, A. et al. BAG3 is involved in neuronal differentiation and migration. Cell Tissue Res. 368, 249-258 (2017).
52. Tang, Y. et al. Fast vesicle transport is required for the slow axonal transport of synapsin. J. Neurosci. 33, 15362-15375 (2013).
53. Ganguly, A. et al. Hsc70 chaperone activity is required for the cytosolic slow axonal transport of synapsin. J. Cell Biol. 216, 04028 (2017).
54. Gamerdinger, M., Kaya, A. M., Wolfrum, U., Clement, A. M. & Behl, C. BAG3 mediates chaperone-based aggresome-targeting and selective autophagy of misfolded proteins. EMBO Rep. 12, 149-156 (2011).
55. Cristofani, R. et al. Inhibition of retrograde transport modulates misfolded protein accumulation and clearance in motoneuron diseases. Autophagy 13, 1280-1303 (2017).
56. Ulbricht, A. & Höhfeld, J. Tension-induced autophagy: may the chaperone be with you. Autophagy 9, 920-922 (2013).
57. Kathage, B. et al. The cochaperone BAG3 coordinates protein synthesis and autophagy under mechanical strain through spatial regulation of mTORC1.
Biochim. et Biophys. Acta 1864, 62-75 (2017).
58. Behl, C. Breaking BAG: the co-chaperone BAG3 in health and disease. Trends Pharmacol. Sci. 37, 672-688 (2016).
59. Curreli, S. et al. B cell lymphoma in HIV transgenic mice. Retrovirology 10, 92 (2013).

### Other Embodiments

From the foregoing description, it will be apparent that variations and modifications may be made to the invention described herein to adopt it to various usages and conditions. Such embodiments are also within the scope of the following claims.

What is claimed is:

1. A composition comprising an agent which inhibits interaction between human immunodeficiency virus (HIV) Tat protein, Bcl-2 associated athanogene (BAG) and/or heat shock protein 70 (HSP70) as measured by a hyperphosphorylated tau accumulation and/or reactive oxygen species in a subject.

2. The composition of claim 1, wherein said agent comprises small molecules, oligonucleotides, polynucleotides, peptides, polypeptides, proteins, gene-editing agents, siRNA, enzymes, peptide nucleic acids, organic molecules, synthetic molecules or combinations thereof.

3. The composition of claim 1, wherein the Bcl-2 associated athanogene (BAG) is BAG3.

4. A method of preventing and/or treating dementia in a subject, comprising: administering to the subject a pharmaceutical composition comprising a therapeutically effective amount of an agent which inhibits interaction between human immunodeficiency virus (HIV) Tat protein, Bcl-2 associated athanogene (BAG) and/or heat shock protein 70 (HSP70), thereby preventing or treating dementia in the subject.

5. The method of claim 4, wherein the subject is infected with HIV.

6. The method of claim 5, wherein the subject is HIV⁺ undetectable.

7. The method of claim 4, wherein the Bcl-2 associated athanogene (BAG) is BAG3.

8. The method of claim 4, wherein the agent decreases hyperphosphorylated tau accumulation in the subject.

9. The method of claim 4, wherein the agent decreases in reactive oxygen species in the subject.

10. The method of claim 4, wherein the agent decreases cell stress and mitochondrial dysfunction in the subject.

11. A method of preventing or treating neurodegeneration in a subject, comprising: administering to the subject a pharmaceutical composition comprising a therapeutically effective amount of an agent which inhibits interaction between human immunodeficiency virus (HIV) Tat protein, Bcl-2 associated athanogene (BAG) and/or heat shock protein 70 (HSP70), thereby preventing or treating dementia in the subject.

12. The method of claim 11, wherein the subject is infected with HIV.

13. The method of claim 11, wherein the subject is HIV⁺ undetectable.

14. The method of claim 11, wherein the Bcl-2 associated athanogene (BAG) is BAG3.

15. The method of claim 11, wherein the agent decreases hyperphosphorylated tau accumulation in the subject.

16. The method of claim 11, wherein the agent decreases in reactive oxygen species in the subject.

17. The method of claim 11, wherein the agent decreases cell stress and mitochondrial dysfunction in the subject.

18. A method of treating preventing or treating neurodegeneration in a subject, comprising administering Bcl2-associated anthanogene 3 (BAG3) polynucleotide, polypeptide and/or agent which induce BAG3 expression or function.

19. A method of preventing and/or treating dementia in a subject, comprising administering a Bcl2-associated anthanogene 3 (BAG3) polynucleotide, polypeptide and/or agent which induce BAG3 expression or function.

20. The method of claims 4, 11, 18 or 19, further comprising administering a second agent as combination therapy.
